SEQUENCE LISTING (6 sequences)

SEQ ID NO: 1 - DNA, 33 bp, Artificial Sequence
  Artificial PCR primer number 1
taggctagccaccatggcttcgtacccctgcca

SEQ ID NO: 2 - DNA, 56 bp, Artificial Sequence
  Artificial PCR primer number 2
gaagcggccgctctagaatcaatgatgatgatgatgatggttagcctcccccatct

SEQ ID NO: 3 - DNA, 50 bp, Artificial Sequence
  Artificial primer number 1 used to create linkers
gcgaattccagcttggcattccggtactgttggtaccatggcgtctagac

SEQ ID NO: 4 - DNA, 50 bp, Artificial Sequence
  Artificial primer number 2 used to create linkers
gcactagtgtattacaatagctaagaatttcgtctagacgccatggtacc

SEQ ID NO: 5 - DNA, 20 bp, Artificial Sequence
  Artificial PCR primer number 3
ccaccatggctagctgtttc

SEQ ID NO: 6 - DNA, 45 bp, Artificial Sequence
  Artificial PCR primer number 4
cgtctagactgaatacagttacatttcaatgatgatgatgatgat

(19) United States
(12) Patent Application Publication
(10) Pub. No.: US 2006/0019922 A1
(43) Pub. Date: Jan. 26, 2006

(54) Selective killing of cancerous cells

(75) Inventors: Rudolph L. Juliano, Chapel Hill, NC (US); Dong Xu, Chapel Hill, NC (US); Vidula Dixit, Chapel Hill, NC (US)

Correspondence Address: JENKINS, WILSON & TAYLOR, P. A., 3100 TOWER BLVD, SUITE 1400, DURHAM, NC, 27707, US

(73) Assignee: The University of North Carolina, Chapel Hill, NC (US)

(21) Appl. No.: 11/181,390

(22) Filed: Jul. 14, 2005

Related U.S. Application Data
(60) Provisional application No. 60/587,748, filed on Jul. 14, 2004.

(51) Int. Cl. A61K 48/00; C12N 15/85

(52) U.S. Cl. 514/44; 435/455

(57) ABSTRACT

The presently disclosed subject matter provides methods for selectively conferring toxicity on cancerous cells. Also provided are systems and kits that can be employed for performing the disclosed method.

### CROSS REFERENCE TO RELATED APPLICATION

This application is based on and claims priority to U.S. Provisional Patent Application Ser. Number 60/587,748, filed Jul. 14, 2004, herein incorporated by reference in its entirety.

### GRANT STATEMENT

This work was supported by National Institutes of Health (NIH) grants RO1-CA77340 and P01 GM-59299. Thus, the U.S. government has certain rights in the presently disclosed subject matter.

### TECHNICAL FIELD

The presently disclosed subject matter generally relates to methods, systems and kits for selectively killing cancerous cells by exploiting the effective absence of functional tumor suppressor proteins in cancerous cells. More particularly, the methods, systems and kits comprise utilization of transcription repressor proteins to regulate genes whose products are capable of conferring toxicity, wherein toxicity is latent unless the cell substantially lacks a functional tumor suppressor protein.

TABLE OF ABBREVIATIONS

| | |
|---|---|
| CB1954 | 5-(aziridin-1-yl)-2,4-dinitrobenzamide |
| CMV | cytomegalovirus |
| DHFR | dihydrofolate reductase |
| E1A | adenovirus early gene 1A |
| E1B | adenovirus early gene 1B |
| E2A | adenovirus early gene 2A |
| E2B | adenovirus early gene 2B |
| E3 | adenovirus early gene 3 |
| E4 | adenovirus early gene 4 |
| GCV | ganciclovir |
| GFP | green fluorescent protein |
| HEK | human embryonic kidney |
| HPRT | hypoxanthine phosphoribosyl transferase |
| hsp | heat shock protein |
| HSV-TK | herpes simplex virus thymidine kinase |
| kb | kilobase |
| KRAB | Kruppel-associated box domain of zif proteins |
| MDR1 | multidrug resistance gene |
| NIH | National Institutes of Health |
| PCR | polymerase chain reaction |
| pfu | plaque forming units |
| PGK | phosphoglycerate kinase |
| PSA | prostate specific antigen |
| RIPA | radioimmunoprecipitation assay |
| Saos-2/p53+ | p53-positive clones |
| Saos-2/p53− | p53-negative clones |
| s-Flt1 | a soluble form of the Flt1 receptor |
| Sp1 | transcription factor |
| SV40 | simian virus 40 |
| TAFs | transcription-associated factors |
| TGF-β | transforming growth factor beta |
| TK | thymidine kinase |
| TKp | thymidine kinase promoter |
| Tm | melting temperature |
| TNF-α | tumor necrosis factor-alpha |
| TRE | transcriptional regulatory element |
| Zif | zinc finger |

### BACKGROUND

Tumor selective cell-killing is a desirable approach to improving cancer therapy, as current cancer therapies are highly toxic and are damaging not only to cancerous cells, but to healthy cells as well. One method considered in tumor selective cell-killing is optimization of the expression of therapeutic genes in tumor cells and minimization of their expression in normal cells. Several strategies attempting this approach have been reported, including direct gene delivery to tumors (Mohr et al., 2001), retroviral integration into rapidly dividing cancer cells (Tamura et al., 1998), and tumor-specific control of transcription (Ido et al., 2001). Targeting tumor cells via the control of transcription has been tested extensively, and several tumor-selective promoters have been identified, such as the hepatoma-associated α-fetoprotein promoter (Ido et al., 2001), the carcinoembryonic antigen promoter in colorectal and lung cancer cells (Kijima et al., 1999), and the tyrosinase gene promoter in melanomas (Siders et al., 1998). These promoters have been used to drive therapeutic genes to selectively kill tumor cells.

A most commonly used therapeutic killing tool is a suicide enzyme/prodrug combination system, wherein the enzymes produced from suicide genes convert nontoxic drugs into cytotoxic compounds. For example, herpes simplex virus thymidine kinase (HSV-TK) and Escherichia coli cytosine deaminase convert ganciclovir and 5-fluorocytosine to the toxic products ganciclovir-triphosphate and 5-fluorouracil, respectively (Ichikawa et al., 2000; Loimas et al., 2001). Although the suicide enzyme/prodrug approach can be powerful and controllable, cell type-specific promoters are relatively weak and are not applicable to many types of tumors, resulting in limits to the efficiency and specificity of the killing.

Tumor suppressor genes are defective in many types of cancers, and approximately 30 tumor suppressor genes have been identified so far in humans (Park & Vogelstein, 2003). The much-studied tumor suppressor p53 is absent or mutated in more than 50% of human tumors (Hainaut, 2002; Lane & Lain, 2002), and abnormalities in the regulation of p53 contribute to cancer (Prives, 1998; Thomas et al., 1999). Thus, several therapeutic strategies have been formulated by evaluating the function and regulation of p53. In some studies, the wild-type p53 gene was delivered to tumor cells, causing apoptosis of the cells in response to cytotoxic drug treatment (Merritt et al., 2001). Another important study (Bischoff et al., 1996) produced a mutant adenovirus that does not express E1B, a protein that binds and inactivates p53. As a result, this mutant virus could replicate in and lyse p53-deficient human tumor cells, but not cells with functional p53 (Heise et al., 1997, 1999a,b).

Other attempts to selectively regulate transcription of cancer related genes have involved the ability to design novel proteins based on a Cys2-His2 type of zinc finger (Zif) DNA binding domain. This ability has allowed the creation of chimeric proteins that have novel DNA sequence binding specificities and strong transcriptional regulatory effects (Beerli et al., 1998; Kim & Pabo, 1998). Novel DNA binding Zifs coupled with transcriptional activator or repressor domains produce strong transcriptional regulatory effects on reporter genes (Kim & Pabo, 1997; Beerli et al., 1998; Kang & Kim, 2000) and on endogenous chromosome-embedded genes (Bartsevich & Juliano, 2000; Beerli et al., 2000a; Kang & Kim, 2000). Previously reported (Bartsevich & Juliano, 2000) was the use of a yeast combinatorial library approach to produce a 5-Zif DNA binding domain directed against a 15-base sequence in the promoter of the MDR1 gene. This was linked to two KRAB-A repressor domains to form K2-5F, a sequence-selective repressor that was designed to regulate the expression of reporter genes driven by the MDR1 promoter sequence. Furthermore, it was also shown that this recombinant transcriptional regulator strongly and selectively repressed the expression of the MDR1 gene in multidrug-resistant human tumor cells (Xu et al., 2002).

Although many attempts have been made to exploit the differences between cancerous cells and non-cancerous cells in order to selectively destroy cancerous cells, there remains much room for improvement in the art. Serious limitations hinder these approaches, including limitations imposed by processes that rely on administration of a therapeutic agent by directly contacting the cancer cells with the therapeutic agent, further compounded by the difficulty in locating and accessing cancer cells in many types of cancers; limitations imposed by processes that use tumor selective promoters for activation of a therapeutic system, wherein many such promoters are either weak-acting or nonexistent, depending on the type of cancer; and limitations imposed by therapeutic dependency on cell characteristics, such as rapidity of cell division, which may not serve as strong distinguishers between cancerous and non-cancerous cells, thus resulting in not enough killing of cancer cells and/or too much killing of non-cancerous cells, depending on dose of therapeutic agent.

Accordingly, there remains a need for therapeutic approaches and tools which are selectively toxic to cancerous cells, are largely not harmful to non-cancerous cells, are applicable to a wide variety of cancer types, and are controllable, efficient, and efficacious. The presently disclosed subject matter addresses these and other needs in the art in whole or in part.

### SUMMARY

The presently disclosed subject matter provides methods, systems and kits for selectively conferring toxicity to cancerous cells, so that the cancerous cells can be killed. In some embodiments, selective killing is achieved through transcriptional control of a gene capable of conferring toxicity.

Thus, in some embodiments, a method of the presently disclosed subject matter comprises providing to a cell: a first nucleic acid encoding a first gene product capable of conferring toxicity to the cell, and a second nucleic acid encoding a second gene product that protects non-cancerous cells from developing toxicity by substantially repressing expression of the first gene product and wherein expression of the second gene product is induced by a protein functionally present in non-cancerous cells but substantially functionally absent in cancerous cells, thereby allowing expression of the first gene product substantially only in cancerous cells.

In some embodiments, a method of the presently disclosed subject matter comprises providing to a cell a first nucleic acid encoding a first gene product that is capable of conferring toxicity to the cell, and a second nucleic acid encoding a second gene product that protects non-cancerous cells from developing toxicity by substantially repressing expression of the first gene product and wherein expression of the second gene product is induced by a protein functionally present in non-cancerous cells but substantially functionally absent in cancerous cells, wherein the toxicity comprises a chemosensitivity, the method further comprising contacting the cell with a chemical agent that interacts with the first gene product, and further wherein an interaction between the chemical agent and the first gene product produces a converted chemical compound that is toxic to the cell, thereby selectively killing cancerous cells having chemosensitivity to the chemical agent.

In some embodiments, a system of the presently disclosed subject matter comprises a first nucleic acid encoding a first gene product that is capable of conferring toxicity to the cell and a second nucleic acid encoding a second gene product that protects non-cancerous cells from developing toxicity by substantially repressing expression of the first gene product and wherein expression of the second gene product is induced by a protein functionally present in non-cancerous cells but substantially functionally absent in cancerous cells, thereby allowing expression of the first gene product substantially only in cancerous cells.

In some embodiments, a system of the presently disclosed subject matter comprises a first nucleic acid encoding a first gene product that is capable of conferring toxicity to the cell, and a second nucleic acid encoding a second gene product that protects non-cancerous cells from developing toxicity by substantially repressing expression of the first gene product and wherein expression of the second gene product is induced by a protein functionally present in non-cancerous cells but substantially functionally absent in cancerous cells, wherein the toxicity comprises a chemosensitivity, the system further comprising a chemical agent that interacts with the first gene product, and further wherein an interaction between the chemical agent and the first gene product produces a converted chemical compound that is toxic to the cell, thereby selectively killing cancerous cells having chemosensitivity to the chemical agent.

In some embodiments, the presently disclosed subject matter provides a kit comprising a first nucleic acid encoding a first gene product that is capable of conferring toxicity to the cell, and a second nucleic acid encoding a second gene product that protects non-cancerous cells from developing toxicity by substantially repressing expression of the first gene product and wherein expression of the second gene product is induced by a protein functionally present in non-cancerous cells but substantially functionally absent in cancerous cells, thereby allowing expression of the first gene product substantially only in cancerous cells.

In some embodiments, the presently disclosed subject matter provides a kit comprising a first nucleic acid encoding a first gene product that is capable of conferring toxicity to the cell, and a second nucleic acid encoding a second gene product that protects non-cancerous cells from developing toxicity by substantially repressing expression of the first gene product and wherein expression of the second gene product is induced by a protein functionally present in non-cancerous cells but substantially functionally absent in cancerous cells, wherein the toxicity comprises a chemosensitivity, the kit further comprising a chemical agent that interacts with the first gene product, and further wherein an interaction between the chemical agent and the first gene product produces a converted chemical compound that is toxic to the cell, thereby selectively killing cancerous cells having chemosensitivity to the chemical agent.

### BRIEF DESCRIPTION OF THE DRAWINGS

FIGS. 1A and 1B are schematic representations depicting methods of introducing constructs of p53-inducible K2-5F and K2-5F-regulated HSV-TK into cells by transfection. The expression of the transcriptional repressor K2-5F is under the control of a p53-responsive promoter. The modified HSV-TK promoter contains sites that bind the K2-5F repressor.

In normal cells (FIG. 1A), wild-type p53 is present and activates the expression of K2-5F, which binds to the TK vector and represses the expression of HSV-TK. In p53-negative cells (FIG. 1B), there is no K2-5F to inhibit the expression of HSV-TK; thus, these cells make ample enzyme and are susceptible to cell killing by GCV.

FIGS. 2A and 2B are schematic representations of the constructs of two vectors used in the methods, systems, and kits of the presently disclosed subject matter.

As shown in FIG. 2A, pFR-2p21-K2-5F produces p53-inducible K2-5F. This vector contains two copies of a p53-binding sequence from the p21 promoter (labeled 2xp21) followed by a TATA box and the K2-5F coding sequence.

As shown in FIG. 2B, pRL-2MDR-TKp-TK produces HSV-TK but is regulated by K2-5F (at the 2x MDR site). This vector contains two copies of a K2-5F binding sequence from the MDR1 promoter followed by the HSV-TK promoter and the coding sequence for HSV-TK. The vector pRL-TKp-TK is the same as pRL-2MDR-TKp-TK, except it lacks the two K2-5F binding sites.

FIG. 3 depicts repression of TK by pcK2-5F as measured by Western blot. Cells (HEK 293T) were co-transfected with equal amounts of the following plasmids: pRL-2MDR-TKp-TK and pcDNA3.1A (lane 1), pRL-2MDR-TKp-TK and pcK2-5F (lane 2), pRL-TKp-TK and pcDNA3.1A (lane 3), pRL-TKp-TK and pcK2-5F (lane 4), or pcDNA3.1A and pRL-TK (lane 5). Expression of HSV-TK and K2-5F was detected by Western blotting using monoclonal anti-His antibody or monoclonal anti-c-myc antibody, respectively.

FIG. 4 is a graphical representation of regulation of the response to GCV by constitutively expressed K2-5F. Cells (HEK 293T) were co-transfected with equal amounts of the following plasmids: pRL-2MDR-TKp-TK and pcDNA3.1A (Δ), pRL-2MDR-TKp-TK and pcK2-5F (◯), or pcDNA3.1A and PRL-TK (□). All cells were also co-transfected with a luciferase-expressing plasmid (FIG. 4A) or a β-galactosidase-expressing plasmid (FIG. 4B) as a marker. After twenty-four hours, cells in each well were re-plated evenly into six-well plates, and in another twenty four hours, GCV was administered in varying concentrations. Luciferase (FIG. 4A) or β-galactosidase (FIG. 4B) activities were measured after four days of treatment by GCV. Results are expressed as a percentage of the enzymatic activities at 0 GCV for each set of plasmids.

FIG. 5 provides a visual representation of repression of TK by p53-induced K2-5F as measured by Western blot. Cells (HEK 293) were co-transfected with the following plasmids: pRL-2MDR-TKp-TK (lane 1), pRL-2MDR-TKp-TK and pFR-2p21-K2-5F (lane 2), pRL-2MDR-TKp-TK, pFR-2p21-K2-5F and pCMV-P53-Myc (lane 3), pRL-2MDR-TKp-TK and pCMV-P53-Myc (lane 4), or pRL-2MDR-TKp-TK and pcK2-5F (lane 5). The total DNA amount was adjusted with pcDNA3.1A and/or PRL-TK. The ratio among pRL-2MDR-TKp-TK, pFR-2p21-K2-5F, and pCMV-P53-Myc was 1:1.5:0.1. Expression of HSV-TK and K2-5F was detected by Western blotting using monoclonal anti-His antibody or monoclonal anti-c-myc antibody, respectively.

FIG. 6 is a graphical representation of regulation of the response to GCV by p53-induced K2-5F. Cells (HEK 293) were co-transfected with the following plasmids: pRL-2MDR-TKp-TK and pFR-2p21-K2-5F (Δ); pRL-2MDR-TKp-TK, pFR-2p21-K2-5F, and pCMV-P53-Myc (∇); or pRL-2MDR-TKp-TK and pcK2-5F (◯). The total DNA amount was adjusted by pcDNA3.1A and/or PRL-TK. A ratio of 1:20:1 between pRL-2MDR-TKp-TK, pFR-2p21-K2-5F, and pCMV-P53-Myc was used. (□) represents cells transfected with both control vectors. After twenty-four hours, cells in each well were replated uniformly into six-well plates, and after another twenty four hours, GCV was administered in varying concentrations. Luciferase activities were measured after four days of treatment with GCV. Results are expressed as a percentage of the luciferase activities at 0 GCV.

FIG. 7 provides a visual representation of the repression of TK by p53-induced K2-5F in Saos-2 cells as measured by Western blot. Cells were cotransfected with the following plasmids: pRL-2MDR-TKp-TK (lane 1); pRL-2MDR-TKp-TK and pFR-2p21-K2-5F (lane 2); pRL-2MDR-TKp-TK, pFR-2p21-K2-5F, and pCMV-P53-Myc (lane 3); or pRL-2MDR-TKp-TK and pCMV-P53-Myc (lane 4). The total DNA amount was adjusted with pcDNA3.1A and/or pRL-TK. Expressed HSV-TK and K2-5F were purified by nickel-bead affinity or immunoprecipitation with monoclonal anti-c-myc antibody and detected by Western blotting using monoclonal anti-His antibody or biotin-labeled monoclonal anti-c-myc antibody, respectively.

FIGS. 8A and 8B provide visual representation of the presently disclosed subject matter as measured by Western blot. FIG. 8A provides representation of the repression of HSV-TK by K2-5F induced by endogenous p53. Saos-2 stable cell lines that were p53-negative (Saos-2/p53-) (lanes 1 and 2) or p53-positive (Saos-2/p53+) (lanes 3 and 4) were transiently transfected with plasmid pRL-2MDR-TKp-TK without (lanes 1 and 3) or with (lanes 2 and 4) co-transfection with pFR-2p21-K2-5F. Expressed HSV-TK was captured by nickel-beads and detected by Western blotting using monoclonal anti-His antibody. Expressed K2-5F and p53 were captured by immunoprecipitation with monoclonal anti-c-myc antibody and detected by Western blotting using biotin-labeled monoclonal anti-c-myc antibody. FIG. 8B depicts the levels of p53 in SAOS-2 stable cell lines versus U-20S osteosarcoma cells. Stably transfected Saos-2/p53+ cells (lane 1) have comparable levels of p53 (top band) as U-20S cells (bottom band) (lane 2). Lane 3 represents U-20S cells transfected with pCMV-p53Myc. p53 was detected by Western blotting using the monoclonal anti-p53 antibody, DO-1. Note that the epitope-tagged p53 runs at a slightly higher position on the gel than native p53.

FIGS. 9A and 9B depict regulation of the response to GCV by K2-5F induced by endogenous p53. Saos-2/p53-negative (FIG. 9A) and Saos-2/p53-positive (FIG. 9B) cells were co-transfected with plasmid pRL-2MDR-TKp-TK without (black bars) or with (gray bars) co-transfection of pFR-2p21-K2-5F. White bars represent cells transfected with empty vectors. All cells were also transfected with a β-galactosidase-expressing plasmid. After twenty-four hours, 600 cells were re-plated in 10-cm plates, and after another twenty-four hours, GCV was administered in varying concentrations. After ten days, surviving blue colonies larger than 50 cells were counted. Survival was expressed as a percentage of colonies formed at 0 GCV. Note that Saos-2 cells require a higher concentration of GCV to attain toxicity than do the HEK 293 cells used in previous figures. The x-axis indicates the concentration of GCV in micromoles. The y-axis indicates the percentage of cell survival compared with cells not treated with GCV.

FIG. 10 provides a visual representation of the expression of TK controlled by a shortened promoter as measured by Western blot. HEK 293T cells were transiently transfected with TK expressing plasmids: control plasmid (lane 1), 0.1 μg, 0.2 μg, 0.5 μg, 0.8 μg, 1.2 μg of pRL-TKp-TK-MIN (lanes 2-6), 0.1 μg, 0.5 μg, 0.8 μg, 1.2 μg of pRL-TKp-TK (lanes 7-11). Final concentration of DNA transfected into each well was brought to 1.2 μg with pcDNA3.1 as filler plasmid. Expression of HSV-TK was detected by monoclonal anti-His antibodies (Covance Research Products, Denver, Pa., United States of America). The arrow indicates the migration position of TK. Note that TK-MIN is a vector with the minimal 200 base TK promoter while TK is a vector with the full length promoter.

FIGS. 11A, 11B, and 11C provide a visual representation of improved repression of TK by CMV promoter-driver K2-5F as measured by Western blot. HEK 293T cells were transiently transfected with the following plasmids: no plasmid control (lane 1), 0.8 μg pRL-2xMDR-1-TKp-TK (top panel, FIG. 11A), 0.8 μg pRL-2xMDR-1-TKp-TK-MIN (middle panel, FIG. 11B), 0.8 μg pRL-6xMDR-1-TKp-TK-MIN (bottom panel, FIG. 11C). Cells were co-transfected with increasing amounts of pcK25F as follows: No pcK25F (lane 2), 0.01 μg, 0.06 μg, 0.12 μg, and 0.2 μg pcK25F (lanes 3-6). Expression of His-tagged HSV-TK was detected by monoclonal anti-His antibodies. The arrow indicates the migration position of TK.

FIGS. 12A and 12B are visual representations of increased production of K2-5F repressor by increasing p53 binding sites on the repressor producing plasmid, as measured by Western blot. Lane 1 is a control. To 0.5 μg of either pFR*-2p21p-K2-5F (FIG. 12A) or pFR*-2p21p-3xMDM2-K2-5F (FIG. 12B) increasing amounts of pCMV-p53 i.e., 0.05 μg, 0.1 μg, 0.2 μg, 0.4 μg (lanes 2-5) were added. Expression of K2-5F and p53 were detected by monoclonal anti-myc antibodies. Arrows indicate the migration position of p53 and K2-5F expression.

FIGS. 13A-13D are visual representations of improved repression of the TK gene by increasing the p53 binding sites on the repressor plasmid at the MDR1 site on the TK plasmid. HEK 293T cells were transiently transfected with the following plasmids: control 1.25 μg pcDNA3.1 (lane 1); lanes 2-5 consists of 0.8 μg pRL-2xMDR-1-TKp-TK-MIN (FIGS. 13A and 13B) and 0.8 μg pRL-6xMDR-1-TKp-TK-MIN (FIGS. 13C and 13D). In lanes 3-5, cells were co-transfected with 0.25 μg pFR*-2p21p-K2-5F (FIGS. 13A and 13C), 0.25 μg pFR*-2p21p-3xMDM2-K2-5F (FIGS. 13B and 13D). Lanes 4 and 5 are cotransfected with 0.05 μg and 0.1 μg PCMV-p53. Expression of HSV-TK was detected by monoclonal anti-His antibodies. Arrows indicate the migration position of TK and tubulin control.

FIGS. 14A and 14B are schematic representations depicting methods of introducing constructs of SMAD4-inducible K2-5F repressor and K2-5F-regulated HSV-TK into cells by transfection. The expression of the transcriptional repressor K2-5F is under the control of a SMAD4-responsive promoter. The modified HSV-TK promoter contains sites that bind the K2-5F repressor.

In normal cells (FIG. 14A), wild-type SMAD4 activates the expression of K2-5F, which binds to the TK vector and represses the expression of HSV-TK.

In SMAD4-negative cells (FIG. 14B), there is no K2-5F to inhibit the expression of HSV-TK; thus, these cells make ample enzyme and are susceptible to cell-killing by GCV.

FIG. 15 is a graphical representation of the TGF-β responsiveness of an IgCα promoter. HepG2 cells were transiently transfected with 1.0 μg pGL3-IgCα-luciferase under serum starved condition. Cells were co-transfected with 1.0 μg each of plasmids expressing either human SMAD3 or SMAD4 or mouse Runx2 proteins either alone or in various combinations. TGF-β at 400 pMoles was added five hours after transfection. Cells with TGF-β are represented by hatched bars; cells without TGF-β are represented by black bars. Luciferase activity was determined forty-eight hours after transfection.

FIG. 16 is a graphical representation of the SMAD responsiveness of an IgCα promoter. “IgCa” refers to the IgCα promoter. AsPC-1 (SMAD4-negative) cells were transiently transfected with 1.0 μg pGL3-IgCα-luciferase. Cells were co-transfected with 1.0 μg each of plasmids expressing either human SMAD3 or SMAD4 or mouse Runx2 proteins either alone or in various combinations as indicated in the figure. Luciferase activity was determined forty-eight hours after transfection.

### DETAILED DESCRIPTION

The presently disclosed subject matter will be now be described more fully hereinafter with reference to the accompanying Examples, in which representative embodiments of the presently disclosed subject matter are shown. The presently disclosed subject matter can, however, be embodied in different forms and should not be construed to be limited to the embodiments set forth herein. Rather, these embodiments are provided so that this disclosure will be thorough and complete, and will fully convey the scope of the presently disclosed subject matter to those skilled in the art.

All publications, patent applications, patents, and other references mentioned herein are incorporated by reference in their entireties.

The presently disclosed subject matter generally relates to methods, systems, and kits for creating a cellular environment of contingent toxicity wherein the absence of a functional tumor suppressor gene product in a cell activates a process resulting in toxicity to the cell, and the presence of the functional tumor suppressor gene product in a cell insulates the cell against the toxicity producing process. Thus, in cancerous cells lacking functional tumor suppressor gene product, the toxicity process is activated and the cells are killed, while in non-cancerous cells, the toxicity producing process remains latent and the cells are unharmed.

I. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

While the following terms are believed to be well understood by one of ordinary skill in the art, the following definitions are set forth to facilitate explanation of the presently disclosed subject matter.

Following long-standing patent law convention, the terms “a”, “an”, and “the” refer to “one or more” when used in this application, including the claims. Thus, for example, reference to “a cell” (e.g., “a HepG2 cell”) includes a plurality of such cells, unless the context clearly is to the contrary (e.g., a plurality of HepG2 cells), and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

As used herein, the term “cell” refers not only to the particular subject cell (e.g., a living biological cell such as a HepG2 cell or a Saos-2 cell), but also to the progeny or potential progeny of such a cell. Because certain modifications can occur in succeeding generations due to either mutation or environmental influences, such progeny might not, in fact, be identical to the parent cell, but are still included within the scope of the term as used herein.

As used herein, the term “kit” refers to a set of articles, implements, or components used for a specific purpose. The presently disclosed subject matter includes kits for the purpose of selectively killing cancerous cells.

As used herein, the term “system” refers to a functionally related group of elements, or a group of interacting components. The presently disclosed subject matter includes systems for the selective killing of cancerous cells.

As used herein, the term “threshold” refers to the point at which a physiological effect begins to be produced. In the presently disclosed subject matter, the physiological effect is either (1) cell death due to toxicity, or (2) lack of toxicity due to repressor activity. The term “substantially” is used herein to indicate threshold status. Thus, if there is cell death due to toxicity, the repressor activity is “substantially functionally absent”, which is to say that although there may be some amount of repressor activity, there is not enough to avoid toxicity.

I.A. Nucleic acids

The term “nucleic acid” refers to deoxyribonucleotides or ribonucleotides and polymers thereof in either single- or double-stranded form. Unless specifically limited, the term encompasses nucleic acids containing known analogues of natural nucleotides that have similar properties as the reference natural nucleic acid. Unless otherwise indicated, a particular nucleotide sequence also implicitly encompasses conservatively modified variants thereof (e.g., degenerate codon substitutions), complementary sequences, subsequences, elongated sequences, as well as the sequence explicitly indicated. The terms “nucleic acid” or “nucleotide sequence” can also be used in place of “gene”, “cDNA”, or “mRNA”. Nucleic acids can be derived from any source, including any organism.

The term “isolated”, as used in the context of a nucleic acid, indicates that the nucleic acid exists apart from its native environment and is not a product of nature. An isolated DNA molecule can exist in a purified form or can exist in a non-native environment such as a transgenic host cell.

The term “gene” refers broadly to any segment of DNA associated with a biological function. A gene typically encompasses one or more sequences selected from the group consisting of a coding sequence, a promoter region, a transcriptional regulatory sequence, a non-expressed DNA segment that is a specific recognition sequence for regulatory proteins, a non-expressed DNA segment that contributes to gene expression, a DNA segment designed to have desired parameters, and combinations thereof. A gene can be obtained by a variety of methods, including isolation or cloning from a biological sample, synthesis based on known or predicted sequence information, and recombinant derivation of an existing sequence. As such, the term “gene” refers to a transcription unit.

In some embodiments, however, the term “gene” is used interchangeably with “coding sequence” and/or “open reading frame” to refer to a protein coding subsequence of a genetic locus. Thus, the term “gene” can be used to refer to an entire genetic locus including a coding sequence, introns, and operatively linked regulatory elements, or it can be used to refer to the coding sequences of a genetic locus without operatively linked regulatory elements (e.g., the exons without or without introns, if present), or it can refer to a spliced or unspliced transcription product of the genetic locus (for example, an mRNA), depending on the context in which the term is employed. In some embodiments, the term “gene” refers to a cDNA.

The term “gene expression” generally refers to the cellular processes by which a biologically active polypeptide is produced from a DNA sequence. Such processes typically include one or more of transcription, splicing, translation, and post-translational processing. In some embodiments, “gene expression” also includes transport of the biologically active polypeptide to an organelle within the cell or to a site outside of the cell.

The presently disclosed subject matter can also employ chimeric genes. The term “chimeric gene”, as used herein, refers to a promoter region operatively linked to a nucleotide sequence encoding a selected polypeptide; a nucleotide sequence producing an antisense RNA molecule; an RNA molecule having tertiary structure, such as a hairpin structure; or a double-stranded RNA molecule. As used herein, the term “mutation” carries its traditional connotation and means a change, inherited, naturally occurring or introduced, in a nucleic acid or polypeptide sequence, and is used in its sense as generally known to those of skill in the art.

The terms “operatively linked” and “operably linked”, as used herein, refer to a promoter region that is connected to a nucleotide sequence (for example, a coding sequence or open reading frame) in such a way that the transcription of the nucleotide sequence is controlled and regulated by that promoter region. Similarly, a nucleotide sequence is said to be under the “transcriptional control” of a promoter to which it is operably linked. Techniques for operatively linking a promoter region to a nucleotide sequence are known in the art.

The term “promoter” or “promoter region” each refers to a nucleotide sequence within a gene that is positioned 5′ to a coding sequence of a same gene and functions to direct transcription of the coding sequence. The promoter region comprises a transcriptional start site, and can additionally include one or more transcriptional regulatory elements. In some embodiments, a method of the presently disclosed subject matter employs a p53 inducible promoter. In some embodiments, a method of the presently disclosed subject matter employs a SMAD4 inducible promoter. In some embodiments, a method of the presently disclosed subject matter employs a WT1 (Wilms tumor) inducible promoter. In some embodiments, a method of the presently disclosed subject matter employs an RB1 inducible promoter.

A “minimal promoter” is a nucleotide sequence that has the minimal elements required to enable basal level transcription to occur. As such, minimal promoters are not complete promoters but rather are subsequences of promoters that are capable of directing a basal level of transcription of a reporter construct in an experimental system. Minimal promoters can be useful in reducing the size of a functional construct so that, for example, the construct will fit into an adenovirus or adeno-virus like vector, for which there is a maximum functional size limit. Minimal promoters include, but are not limited to the CMV minimal promoter, the HSV-TK minimal promoter, the simian virus 40 (SV40) minimal promoter, the human Mactin minimal promoter, the human EF2 minimal promoter, the adenovirus E1B minimal promoter, and the heat shock protein (hsp) 70 minimal promoter. In some embodiments of the presently disclosed subject matter, an HSV-TK promoter was shortened by 531 nucleotide base pairs, thereby making it more suitable for transfer into viral vector. See Example 15.

Minimal promoters are often augmented with one or more transcriptional regulatory elements to influence the transcription of an operably linked gene. For example, cell-type-specific or tissue-specific transcriptional regulatory elements can be added to minimal promoters to create recombinant promoters that direct transcription of an operably linked nucleotide sequence in a cell-type-specific or tissue-specific manner.

Different promoters have different combinations of transcriptional regulatory elements. Whether or not a gene is expressed in a cell is dependent on a combination of the particular transcriptional regulatory elements that make up the gene's promoter and the different transcription factors that are present within the nucleus of the cell. As such, promoters are often classified as “constitutive”, “tissue-specific”, “cell-type-specific”, or “inducible”, depending on their functional activities in vivo or in vitro. For example, a constitutive promoter is one that is capable of directing transcription of a gene in a variety of cell types. Exemplary constitutive promoters include the promoters for the following genes which encode certain constitutive or “housekeeping” functions: hypoxanthine phosphoribosyl transferase (HPRT), dihydrofolate reductase (DHFR; Scharfmann et al., 1991), adenosine deaminase, phosphoglycerate kinase (PGK), pyruvate kinase, phosphoglycerate mutase, the β-actin promoter (see e.g., Williams et al., 1993), and other constitutive promoters known to those of skill in the art. “Tissue-specific” or “cell-type-specific” promoters, on the other hand, direct transcription in some tissues and cell types but are inactive in others. Exemplary tissue-specific promoters include the PSA promoter (Yu et al., 1999; Lee et al., 2000), the probasin promoter (Greenberg et al., 1994; Yu et al., 1999), and the MUC1 promoter (Kurihara et al., 2000), as well as other tissue-specific and cell-type specific promoters known to those of skill in the art.

An “inducible” promoter is one for which the transcription level of an operably linked gene varies based on the presence of a certain stimulus. Genes that are under the control of inducible promoters are expressed only, or to a greater degree, in the presence of an inducing agent, (e.g., transcription under control of the metallothionein promoter is greatly increased in presence of certain metal ions). Inducible promoters include transcriptional regulatory elements (TREs), which stimulate transcription when their inducing factors are bound. For example, there are TREs for serum factors, steroid hormones, retinoic acid and cyclic AMP. Promoters containing a particular TRE can be chosen in order to obtain an inducible response, and in some cases, the TRE itself can be attached to a different promoter, thereby conferring inducibility to the recombinant gene. Inducible promoters that can be employed in the instantly disclosed systems, methods, and kits include, but are not limited to promoters responsive to a tumor suppressor protein involved in transcription, such as p53, SMAD4, RB1, or WT1.

When used in the context of a promoter, the term “linked” as used herein refers to a physical proximity of promoter elements such that they function together to direct transcription of an operably linked nucleotide sequence. In some embodiments of the presently disclosed subject matter, a nucleic acid comprises a tumor suppressor protein-inducible promoter that is linked to transcription of a repressor gene. In some embodiments, a nucleic acid comprises a p53 inducible promoter that is linked to transcription of a K2-5F repressor gene. In some embodiments, a nucleic acid comprises an SMAD4 inducible promoter that is linked to transcription of a K2-5F repressor gene.

The term “transcriptional regulatory sequence” or “transcriptional regulatory element”, as used herein, refers to a nucleotide sequence within the promoter region that enables responsiveness to a regulatory transcription factor. Responsiveness can encompass a decrease or an increase in transcriptional output and is mediated by binding of the transcription factor to the DNA molecule comprising the transcriptional regulatory element.

The term “transcription factor” generally refers to a protein that modulates gene expression by interaction with the transcriptional regulatory element and cellular components for transcription, including RNA Polymerase, Transcription Associated Factors (TAFs), chromatin-remodeling proteins, and any other relevant protein that impacts gene transcription.

The terms “reporter gene”, “marker gene”, and “selectable marker” refer to a heterologous gene encoding a product that is readily observed and/or quantitated. A reporter gene is heterologous in that it originates from a source foreign to an intended host cell or, if from the same source, is modified from its original form. Non-limiting examples of detectable reporter genes that can be operatively linked to a transcriptional regulatory region can be found in Alam & Cook (1990) and PCT International Publication No. WO 97/47763. Exemplary reporter genes for transcriptional analyses include the lacZ gene (see e.g., Rose & Botstein (1983)), Green Fluorescent Protein (GFP; Cubitt et al., 1995), luciferase, and chloramphenicol acetyl transferase (CAT). Reporter genes for methods to produce transgenic animals include, but are not limited to antibiotic resistance genes, for example the antibiotic resistance gene confers neomycin resistance. Any suitable reporter and detection method can be used, and it will be appreciated by one of skill in the art that no particular choice is essential to or a limitation of the presently disclosed subject matter.

An amount of reporter gene expression can be assayed by any method for qualitatively or quantitatively determining the presence or activity of the reporter gene product. The amount of reporter gene expression directed by each test promoter region fragment is compared to an amount of reporter gene expression to a control construct comprising the reporter gene in the absence of a promoter region fragment. A promoter region fragment is identified as having promoter activity when there is significant increase in an amount of reporter gene expression in a test construct as compared to a control construct. The term “significant increase”, as used herein, refers to a quantified change in a measurable quality that is larger than the margin of error inherent in the measurement technique, in some embodiments an increase by about 2-fold or greater relative to a control measurement, in some embodiments an increase by about 5-fold or greater, and in some embodiments an increase by about 10-fold or greater.

The term “vector”, as used herein refers to a DNA molecule having sequences that enable the transfer of those sequences to a compatible host cell. A vector also includes nucleotide sequences to permit ligation of nucleotide sequences within the vector, wherein such nucleotide sequences are also replicated in a compatible host cell. A vector can also mediate recombinant production of a selected polypeptide, as described further herein below. As used herein, the terms “vector” and “plasmid” are interchangeable.

Nucleic acids of the presently disclosed subject matter can be cloned, synthesized, recombinantly altered, mutagenized, or combinations thereof. Standard recombinant DNA and molecular cloning techniques used to isolate nucleic acids are known in the art. Exemplary, non-limiting methods are described by Silhavy et al., 1984; Ausubel et a., 1992; Glover & Hames, 1995; and Sambrook & Russell, 2001). Site-specific mutagenesis to create base pair changes, deletions, or small insertions is also known in the art as exemplified by publications (see e.g., Adelman et al., 1983; Sambrook & Russell, 2001).

I.B. Gene Products or Polypeptides

The terms “gene product” is herein used interchangeably with the term “polypeptide”. The gene products employed in accordance with the presently disclosed subject matter include all embodiments comprising biologically functional fragments, including those that comprise a fusion protein. The gene products employed in accordance with the presently disclosed subject matter include, but are not limited to isolated gene products, gene products that are polypeptide fragments, gene products that are full length polypeptides, gene products that comprise fusion proteins, biologically functional analogs, and gene products that cross-react with an antibody that specifically recognizes a disclosed gene product.

The term “isolated”, as used in the context of a gene product, indicates that the gene product exists apart from its native environment and is thus not a product of nature. An isolated gene product can exist in a purified form or can exist in a non-native environment such as, for example, in a transgenic host cell.

The methods of the presently disclosed subject matter can employ gene product or polypeptide fragments or functional portions of a gene product or polypeptide, such as a repressor gene product. Such functional portion need not comprise all or substantially all of the amino acid sequence of a native gene product. The term “functional” includes any biological activity or feature of the polypeptide.

The presently disclosed subject matter also includes longer sequences of a gene product or polypeptide. For example, one or more amino acids can be added to the N-terminus or C-terminus of the polypeptide. Fusion proteins (for example, repressor polypeptide sequences) are also provided within the scope of the presently disclosed subject matter. Methods of preparing such proteins are known in the art. In one example, the fusion protein includes any biological activity of a selected gene product or polypeptide. In the case of a repressor polypeptide, the biological activity is in some embodiments any biological activity of a repressor protein. Optionally, a fusion protein can have additional biological activities provided by the fused heterologous sequence. Included herein are gene products or polypeptides wherein a repressor protein is fused with a steroid such as estrogen, to control entry of the repressor polypeptide into the cell's nucleus, thus controlling the activity of the repressor. The repressor in such a system cannot enter the nucleus until it contacted by a drug, such as tamoxifen, which is effective if the steroid is estrogen.

The presently disclosed subject matter also encompasses functional analogs of a selected polypeptide. Functional analogs share at least one biological function with a selected polypeptide (for example, a repressor polypeptide). In the context of amino acid sequence, biologically functional analogs, as used herein, are peptides in which certain, but not most or all, of the amino acids can be substituted. Functional analogs can be created at the level of the corresponding nucleic acid molecule, altering such sequence to encode desired amino acid changes. In some embodiments, changes can be introduced to improve a biological function of the polypeptide, e.g., to improve the desire effect of the polypeptide (for example, a repressor polypeptide).

The presently disclosed subject matter also encompasses recombinant production of the disclosed polypeptides. Briefly, a nucleic acid sequence encoding a selected polypeptide is cloned into a construct, and the construct is introduced into a host organism, where it is recombinantly produced. The term “host organism” refers to any organism into which a disclosed vector has been introduced. In some embodiments, the host organism is a warm-blooded vertebrate; in some embodiments, a mammal.

II. Methods, Systems, and Kits

Selective killing of tumor cells is highly desirable in cancer therapeutics. One effective way to distinguish between cancerous cells and non-cancerous cells is to determine the presence or absence of functional tumor suppressor proteins, which are found in non-cancerous cells but are missing from many types of cancerous cells. For example, the tumor suppressor transcription factor p53 is absent or mutated in more than 50% of human tumors. Therefore, determining treatment approaches that utilize the status (presence or absence) of functional tumor suppressor proteins, like p53, to regulate therapy is an important strategy in attaining therapeutic cancer cell selectivity. The methods, systems, and kits of the presently disclosed subject matter provide selective killing of tumor cells by utilizing the presence of functional tumor suppressor proteins, such as but not limited to p53, in combination with transcriptional repressor proteins, such as but not limited to K2-5F.

Thus, in some embodiments, a method of the presently disclosed subject matter comprises providing to a cell a first nucleic acid encoding a first gene product that is capable of conferring toxicity to the cell and a second nucleic acid encoding a second gene product that protects non-cancerous cells from developing toxicity by substantially repressing expression of the first gene product and wherein expression of the second gene product is induced by a protein functionally present in non-cancerous cells but substantially functionally absent in cancerous cells, thereby allowing expression of the first gene product.

The first nucleic acid of the presently disclosed subject matter can encode a first gene product that confers direct toxicity on the cell when present in a cell or the first nucleic acid can encode a first gene product that confers indirect toxicity on the cell when present in the cell in conjunction with a chemical agent that interacts with the first gene product, resulting in the formation of a product toxic to the cell. Exemplary gene products capable of conferring direct toxicity to a cell include, but are not limited to, TNF-α, TNF-β, FasL, APRIL, BAFF, OPGL/TRANCE/RANCL, TALL-1, TRAIL, any other TNF family members (as identified by protein structure and/or function), Bad, Bax, Bak, Bcl-xₛ, Bok/Mtd, Bcl-G_{L}, Bik/Nbk, Blk, Bid, Hrk/DP5, Bim/Bod, Bmf, Noxa, Puma/Bbc-3 (Wu & Deng, 2002), C. elegans Egl-1; toxic subunits of plant or bacterial toxins, such as ricin, abrin, or Diphtheria toxin (Olsnes, 2004; Johannes & Decaudin, 2005); or cytotoxic peptides such as mellitin (Ling et al., 2005). Exemplary gene products capable of conferring indirect toxicity to a cell include, but are not limited to, HSV-TK, which interacts with the chemical agent ganciclovir to produce toxicity; cytosine deaminase, which interacts with the chemical agent 5-fluorocytosine to produce toxicity; nitroreductase, which interacts with the chemical agent CB1954 to produce toxicity; and carboxypeptidase, which interacts with the chemical agent CMDA to produce toxicity (El-Aneed, 2004).

The second nucleic acid of the presently disclosed subject matter encodes a second gene product that represses the expression of the first gene product that is capable of conferring toxicity to the cell in which it is expressed. A gene product that represses expression of another gene can be referred to as a “repressor”. In some embodiments, the second gene product of the presently disclosed subject matter is comprised of at least one repressor domain and at least two DNA binding domains, as described more fully below. In some embodiments, the second gene product comprises two, three, four, five, six, or more zinc finger DNA binding domains. In some embodiments, the second gene product comprises two zinc finger DNA binding domains. In some embodiments, the second gene product comprises three zinc finger DNA binding domains. In some embodiments, the second gene product comprises four zinc finger DNA binding domains. In some embodiments, the second gene product comprises five zinc finger DNA binding domains. In some embodiments, the second gene product comprises six zinc finger DNA binding domains. In some embodiments, the second gene product comprises seven zinc finger DNA binding domains. In some embodiments, the second gene product comprises eight zinc finger DNA binding domains. In some embodiments, the second gene product comprises nine zinc finger DNA binding domains. In some embodiments, the second gene product comprises ten zinc finger DNA binding domains. In some embodiments, more than ten zinc finger DNA binding domains are contemplated. A number of Zifs effective in various embodiments of the methods, systems, and kits of the currently disclosed subject matter can be determined by one of ordinary skill in the art.

Recent advances in the design, selection, and engineering of DNA binding proteins have led to the emerging field of designer transcription factors (TFs). Modular DNA-binding protein domains can be assembled to recognize a given sequence of a DNA in a regulatory region of a targeted gene. TFs can be readily prepared by linking a nucleotide sequence encoding the DNA-binding protein to nucleic acid sequences encoding a variety of effector domains that mediate transcriptional activation or repression.

Furthermore, the interaction between the TF and the genomic DNA can be regulated by several approaches, including chemical regulation by a variety of small molecules. Genome-wide single target specificity has been demonstrated using arrays of sequence-specific zinc finger (Zif) domains, sometimes referred to as polydactyl proteins. After a review of the present disclosure, a person of ordinary skill in the art can construct polydactyl Zif proteins without undue experimentation by generating a nucleic acid molecule that links coding sequences that encode predefined Zif units that recognize specific triplets of DNA. It is recognized that the action of designed transcriptional repressors is specific and provides high binding affinity, such that designed transcriptional regulators are used to strongly and selectively influence expression of cancer-related genes, even under circumstances of extensive amplification of the target gene.

Thus, after review of the instant specification, those of ordinary skill in the art can design a repressor that can be employed with the methods, systems, and kits of the presently disclosed subject matter. Such repressors are intended to be encompassed by the scope of the presently disclosed subject matter. For example, a representative designed repressor that can inhibit transcription of the first nucleic acid includes, but is not limited to, the K2-5F gene product (Xu et al., 2002).

To elaborate, K2-5F was developed using a yeast combinatorial library approach to produce a 5-Zif DNA binding domain directed against a 15-base sequence in the promoter of the MDR1 gene. This was linked to two KRAB-A repressor domains to form K2-5F, a designed sequence-selective repressor that regulated the expression of reporter genes driven by the MDR1 promoter sequence (Xu et al., 2002).

Proteins “functionally present” in non-cancerous cells but “substantially functionally absent” in cancerous cells include tumor repressor proteins that affect transcription in the cell. The term “substantially functionally absent” includes circumstances wherein the DNA encoding the protein is not transcribed at all, the DNA encoding the protein is transcribed but not translated, or the DNA is transcribed and translated but has some sort of defect that interferes with its ability to fold, bind, and/or retain its biological function. “Substantially functionally absent” thus includes any instance in which the protein is absent or defective such that its ability to be active at normal physiological levels within the cell is destroyed. The term “substantially functionally absent” also refers to the fact that although there may be some amount of DNA, polypeptide, or protein present in the cell, it is not a sufficient amount to allow the cell to reach the threshold level of protection from toxicity due to sufficient repressor activity. The term “functionally present” includes any circumstance wherein the biological activity of the tumor suppressor protein is sufficiently present to avoid cell toxicity, including instances in which the functionality comes from an active polypeptide or gene product fragment and not the full-length protein product.

In some embodiments, a protein that is functionally present in a non-cancerous cell but functionally absent in a cancerous cell is a tumor suppressor protein. Examples of tumor repressor proteins include, but are not limited to, p53, RB1, SMAD4 and WT1. p53 is associated with brain tumors, colon tumors, sarcomas, leukemia, and breast cancer, and is active in cell cycle regulation and apoptosis. RB1 is associated with retinoblastoma and osteogenic sarcoma, and is involved in cell cycle regulation. SMAD4 (the human homolog of the Drosophila gene known as DPC4) is associated with pancreatic carcinoma and colon cancer, and is involved with regulation of TGFβ signal transduction. WT1 is associated with pediatric kidney cancer and is involved in transcriptional regulation. Those of ordinary skill in the art will understand that any protein with tumor suppressing characteristics that is involved in transcription can be utilized in the methods, systems, and kits of the presently disclosed subject matter.

Expression of the second nucleic acid is induced by the protein functionally present in non-cancerous cells but substantially functionally absent in cancerous cells. Further levels of transcriptional manipulation and control are added to the presently disclosed subject matter by using drug-responsive systems. The term “drug-responsive system” includes, but is not limited to, a system that utilizes a drug-inducible gene expression system or a system in which the repressor is fused to a hormone receptor and is thus controlled by a drug that acts on the hormone receptor. Such drug-responsive systems for induction include a Tet-On or Tet-Off system (such as, but not limited to, the one sold by BD Biosciences, San Jose, Calif., United States of America), wherein the promoter inducible by the presence of a tumor suppressor is also under the control of a tetracycline (Tet)-inducible gene expression system. In a Tet-off system, induction of gene expression occurs when Tet (or a tetracycline derivative, such as doxycycline (Dox)) is removed from the cell (Beerli et al., 2000a). In a Tet-on system, induction of gene expression occurs when Tet (or Dox) is added to the cell. Drug-responsive systems also include systems comprised of ligand-dependent transcriptional regulators that are ideally activated by a small molecule inducer with no other biological activity (the drug) that binds to specific sequences present only in the target promoter, and have low immunogenicity. An example of such a system is one wherein the function of the expressed repressor protein is controlled by fusing the repressor and the ligand-binding domain of the estrogen/tamoxifen receptor. This system prevents the repressor from entering the nucleus unless tamoxifen is present (Beerli et al., 2000b). Thus, in the presence of tamoxifen, the repressor is activated and cells containing functional tumor suppressor protein are protected from the toxicity process.

The methods, systems, and kits of the presently disclosed subject matter operate within what can be thought of as a threshold-driven process. Essentially, there is sufficient protection provided by the repressor to prevent the toxicity process from killing the cell, and/or there is sufficient activation of the toxicity process so that the cell is killed. The inclusion of drug-responsive systems for further levels of transcriptional manipulation and control can serve to push cells to one side of the threshold or the other. Stated another way, the further levels of transcriptional manipulation and control can provide either additional repressor to protect the cell from death, or can allow additional expression of the first gene product so that toxicity is activated and the cell is killed.

Thus, in some embodiments, the presently disclosed subject matter comprises providing to a cell: a first nucleic acid encoding a first gene product capable of conferring toxicity to the cell and a second nucleic acid encoding a second gene product that protects non-cancerous cells from developing toxicity by substantially repressing expression of the first gene product and wherein expression of the second gene product is induced by a protein functionally present in non-cancerous cells but functionally absent in cancerous cells, thereby allowing expression of the first gene product, the method further comprising providing to the cell a drug-responsive system that enhances the conference of toxicity to a cancerous cell and/or enhances protection of a non-cancerous cell from toxicity.

In some embodiments of the presently disclosed subject matter that utilize a chemical agent, a suicide gene/prodrug combination can be used. Therefore, in some embodiments, the presently disclosed subject matter provides methods for providing to a cell: a first nucleic acid encoding a first gene product that is capable of conferring toxicity to the cell and a second nucleic acid encoding a second gene product that protects non-cancerous cells from developing toxicity by substantially repressing expression of the first gene product and wherein expression of the second gene product is induced by a protein functionally present in non-cancerous cells but substantially functionally absent in cancerous cells, wherein the toxicity is a chemosensitivity to a chemical agent that recognizes the first gene product and further wherein an interaction between the chemical agent and the first gene product results in a converted chemical compound that is toxic to the cell, thereby selectively killing cancerous cells having chemosensitivity to the chemical agent. In some embodiments, the first gene product is an HSV-TK polypeptide and the chemical agent is ganciclovir. In some embodiments, the second gene product is a K2-5F polypeptide. In some embodiments, the protein functionally present in non-cancerous cells but substantially functionally absent in cancerous cells is a p53 protein. In some embodiments, the protein functionally present in non-cancerous cells but substantially functionally absent in cancerous cells is an SMAD4 protein. In some embodiments, the protein functionally present in non-cancerous cells but substantially functionally absent in cancerous cells is an RB1 protein. In some embodiments, the protein functionally present in non-cancerous cells but substantially functionally absent in cancerous cells is a WT1 protein.

The suicide gene/prodrug strategy can rely upon intracellular conversion of a relatively nontoxic prodrug into a toxic drug by an enzyme of xenobiotic origin, and has been referred to as genetic prodrug activation therapy (GPAT). Plant, fungi, bacteria and viruses often utilize unique metabolic pathways that are adapted to their life cycles and environments. These metabolic routes are not used by mammalian cells. In the case of pathogen infections, the distinctive enzymes responsible for these functions have been the target of prodrugs that are developed to be selectively metabolized in infected cells, leading to their destruction. This process is selective because the prodrug is not toxic to healthy, uninfected mammalian cells that typically lack the enzymes required to transform the prodrug into the toxic metabolite (Vassaux & Lemoine, 2000).

The transfer of the genes encoding these enzymes to mammalian cells is sufficient to confer sensitivity to particular prodrugs that are metabolized by the individual enzymes. In terms of cancer gene therapy, the process involves targeted delivery of these genes to the cancer cells, followed by the administration of the prodrug. Compared with a more direct approach, using a suicide gene such as the diphtheria toxin A chain, this prodrug-enzyme system offers extra levels of control in terms of variation of prodrug concentration as well as prodrug bioavailability and tissue distribution. In some embodiments of the presently disclosed subject matter, a further selective step is added in that the toxicity process is activated only in the absence of a functional tumor suppressing protein. Also in some embodiments of the presently disclosed subject matter, yet another level of control is added in instances wherein a Tet-on/off system or a steroid fusion/drug system is utilized.

Thus, the presently disclosed subject matter provides, in some embodiments, (1) reliance on a lack of tumor suppressor functionality in a cell for activation of a toxicity system and (2) reliance on a lack of repressor activity before the gene product capable of conferring toxicity can be produced. In some embodiments, the presently disclosed subject matter provides (1) reliance on lack of tumor suppressor functionality in a cell for activation of a toxicity system, (2) reliance on a lack of repressor activity before the gene product capable of conferring toxicity can be produced, and (3) reliance on the presence of a chemical agent before toxicity to the cells can be carried to completion. The various embodiments of the presently disclosed subject matter offer safe, efficacious, and controllable methods, systems, and kits for selective killing of cancer cells.

In some embodiments, the enzymes employed for use as prodrugs are monomeric proteins, without any requirement for glycosylation. More complex enzymes might not be correctly folded in an ectopic environment and, as a result, might be less efficient at converting the prodrug. This conversion should be rapid {i.e., have a high coefficient of catalysis (Kcat)} and should require low concentrations of prodrug {i.e., a low Michaelis-Menten constant (Km)}. In addition, the prodrug should be ideally at least two orders of magnitude less toxic than the active anabolite. Several enzyme/prodrug systems are known, and include, but are not limited to, HSV-TK with ganciclovir or acyclovir; cytosine deaminase with 5-fluorocytosine; and nitroreductase with 5-(aziridin-1-yl)-2,4-dinitrobenzamide.

Ganciclovir and acyclovir are guanosine analogues that are poorly metabolized by mammalian cellular thymidine kinases. By contrast, herpes simplex virus thymidine kinase (HSV-TK) metabolizes these prodrugs very efficiently to their monophosphate forms (the rate-limiting step). In turn, the monophosphate form is metabolized to ganciclovir diphosphate and triphosphate by cellular enzymes. The triphosphate form of the prodrug inhibits α-DNA polymerase and is incorporated into DNA, resulting in chain termination during replication. This molecular mechanism of action implies that this enzyme-prodrug system might only be effective for actively dividing cells and should not affect quiescent cells within a tumor. This view has been challenged by the observation that this system induces significant cell death in tissues with low mitotic indices, however. In these cases, the mechanism of action is unclear and seems to involve a p53-independent apoptosis. Very recently, a library of mutants of HSV-TK was created from which a more effective form of the enzyme (mutant 30) was described. This type of approach could be applied to improve the characteristics of other enzymes.

Cytosine deaminase (CD) is an enzyme of bacterial or fungal origin that is activated in response to nutritional stress, deaminating cytosine to uracil. This enzyme became a target for therapy and the prodrug 5-fluorocytosine was selected. 5-Fluorocytosine is metabolized to 5-fluorouracil by CD. Further metabolism of 5-fluorouracil to 5-fluorouridine-5′-triphosphate and 5-fluoro-2′-deoxyuridine-5′-monophosphate results in cell death by affecting RNA and DNA synthesis. The cytotoxic action of CD/5-fluorocytosine requires the proliferation of the target cell. Moreover, 5-fluorouracil is used as a single agent in a limited number of cancers (gastrointestinal tract, for example).

Nitroreductase is a monomeric enzyme that converts non-toxic monofunctional alkylating agents to their difunctional forms. The products of these reactions are four orders of magnitude more toxic than the substrates. Escherichia coli nitroreductase has been used to metabolize the prodrug 5-(aziridin-1-yl)-2,4-dinitrobenzamide to 5-(aziridin-1-yl)-4-(hydroxyamino)-2-nitrobenzamide, in the presence of cellular nicotinamide adenine dinucleotide, reduced form, or nicotinamide adenine dinucleotide phosphate, reduced form, acting as a reductant. The metabolized prodrug is then acetylated to 5-(aziridin-1-yl)-4-(acetylamino)-2-nitrobenzamide, which is capable of cross-linking cellular DNA, resulting in apoptosis. This system offers a large number of potential prodrugs that can be optimized. The prodrug currently used in conjunction with nitroreductase is CB1954. This system offers an advantage over HSV-TK and CD in that it does not require cell proliferation to induce cell death.

In some embodiments, a system of the presently disclosed subject matter comprises a first nucleic acid encoding a first gene product that is capable of conferring toxicity to the cell, and a second nucleic acid encoding a second gene product that protects non-cancerous cells from developing toxicity by substantially repressing expression of the first gene product and wherein expression of the second gene product is induced by a protein functionally present in non-cancerous cells but substantially functionally absent in cancerous cells, thereby allowing expression of the gene product capable of conferring toxicity substantially only in cancerous cells. In some embodiments, the system further comprises a pharmaceutically or physiologically acceptable carrier. In some embodiments, the first gene product is selected from the group consisting of TNF-α, TNF-β, FasL, APRIL, BAFF, OPGL/TRANCE/RANCL, TALL-1, TRAIL, any other TNF family members (as identified by protein structure and/or function), Bad, Bax, Bak, Bcl-x_{S}, Bok/Mtd, Bcl-G_{L}, Bik/Nbk, Blk, Bid, Hrk/DP5, Bim/Bod, Bmf, Noxa, Puma/Bbc-3, C. elegans Egl-1, ricin, abrin, Diptheria toxin, and mellitin. In some embodiments, the second gene product is a K2-5F polypeptide. In some embodiments, the protein functionally present in non-cancerous cells but substantially functionally absent in cancerous cells is a p53 protein. In some embodiments, the protein functionally present in non-cancerous cells but substantially functionally absent in cancerous cells is an SMAD4 protein. In some embodiments, the protein functionally present in non-cancerous cells but substantially functionally absent in cancerous cells is an RB1 protein. In some embodiments, the protein functionally present in non-cancerous cells but substantially functionally absent in cancerous cells is a WT1 protein.

In some embodiments, a system of the presently disclosed subject matter comprises a first nucleic acid encoding a first gene product that is capable of conferring toxicity to the cell and a second nucleic acid encoding a second gene product that protects non-cancerous cells from developing toxicity by substantially repressing expression of the first gene product and wherein expression of the second gene product is induced by a protein functionally present in non-cancerous cells but substantially functionally absent in cancerous cells, wherein the toxicity is a chemosensitivity to a chemical agent that interacts with the first gene product, resulting in a converted chemical compound that is toxic to the cell, thereby selectively killing cancerous cells having chemosensitivity to the chemical agent. In some embodiments, the system further comprises a pharmaceutically or physiologically acceptable carrier. In some embodiments, the first gene product is an HSV-TK polypeptide and the chemical agent is ganciclovir. In some embodiments, the second gene product is a K2-5F polypeptide. In some embodiments, the protein functionally present in non-cancerous cells but substantially functionally absent in cancerous cells is a p53 protein. In some embodiments, the protein functionally present in non-cancerous cells but substantially functionally absent in cancerous cells is an SMAD4 protein. In some embodiments, the protein functionally present in non-cancerous cells but substantially functionally absent in cancerous cells is an RB1 protein. In some embodiments, the protein functionally present in non-cancerous cells but substantially functionally absent in cancerous cells is a WT1 protein.

In some embodiments, the presently disclosed subject matter provides a kit comprising a first nucleic acid encoding a first gene product that is capable of conferring toxicity to the cell, and a second nucleic acid encoding a second gene product that protects non-cancerous cells from developing toxicity by substantially repressing expression of the first gene product and wherein expression of the second gene product is induced by a protein functionally present in non-cancerous cells but substantially functionally absent in cancerous cells, thereby allowing expression of the first gene product only in cancerous cells. In some embodiments, the system further comprises a pharmaceutically or physiologically acceptable carrier. In some embodiments, the first gene product is selected from the group consisting of TNF-α, TNF-β, FasL, APRIL, BAFF, OPGL/TRANCE/RANCL, TALL-1, TRAIL, any other TNF family members (as identified by protein structure and/or function), Bad, Bax, Bak, Bcl-x_{S}, Bok/Mtd, Bcl-G_{L}, Bik/Nbk, Blk, Bid, Hrk/DP5, Bim/Bod, Bmf, Noxa, Puma/Bbc-3, C. elegans Egl-1, ricin, abrin, Diptheria toxin, and mellitin. In some embodiments, the second gene product is a K2-5F polypeptide. In some embodiments, the protein functionally present in non-cancerous cells but substantially functionally absent in cancerous cells is a p53 protein. In some embodiments, the protein functionally present in non-cancerous cells but substantially functionally absent in cancerous cells is an SMAD4 protein. In some embodiments, the protein functionally present in non-cancerous cells but substantially functionally absent in cancerous cells is an RB1 protein. In some embodiments, the protein functionally present in non-cancerous cells but substantially functionally absent in cancerous cells is a WT1 protein.

In some embodiments, the presently disclosed subject matter provides a kit comprising a first nucleic acid encoding a first gene product that is capable of conferring toxicity to the cell, and a second nucleic acid encoding a second gene product that protects non-cancerous cells from developing toxicity by substantially repressing expression of the first gene product and wherein expression of the second gene product is induced by a protein functionally present in non-cancerous cells but substantially functionally absent in cancerous cells, wherein the toxicity is a chemosensitivity to a chemical agent that interacts with the first gene product, resulting in a converted chemical compound that is toxic to the cell, thereby selectively killing cancerous cells having chemosensitivity to the chemical agent. In some embodiments, the first gene product is an HSV-TK polypeptide and the chemical agent is ganciclovir. In some embodiments, the gene product of the second nucleic acid is a K2-5F polypeptide. In some embodiments, the protein functionally present in non-cancerous cells but substantially functionally absent in cancerous cells is a p53 protein. In some embodiments, the protein functionally present in non-cancerous cells but substantially functionally absent in cancerous cells is an SMAD4 protein. In some embodiments, the protein functionally present in non-cancerous cells but substantially functionally absent in cancerous cells is an RB1 protein. In some embodiments, the protein functionally present in non-cancerous cells but substantially functionally absent in cancerous cells is a WT1 protein.

Thus, in some embodiments, the methods, systems, and kits of the presently disclosed subject matter comprise a first nucleic acid encoding a first gene product that is capable of conferring toxicity to the cell, and a second nucleic acid encoding a second gene product that protects non-cancerous cells from developing toxicity by substantially repressing expression of the first gene product and wherein expression of the second gene product is induced by a protein functionally present in non-cancerous cells but substantially functionally absent in cancerous cells, thereby allowing expression of the first gene product, wherein the first gene product is a Bax polypeptide, the second gene product is a K2-5F polypeptide, and the protein functionally present in non-cancerous cells but substantially functionally absent in cancerous cells is a p53 protein.

In some embodiments, the methods, systems, and kits of the presently disclosed subject matter comprise a first nucleic acid encoding a first gene product that is capable of conferring toxicity to the cell, and a second nucleic acid encoding a second gene product that protects non-cancerous cells from developing toxicity by substantially repressing expression of the first gene product and wherein expression of the second gene product is induced by a protein functionally present in non-cancerous cells but substantially functionally absent in cancerous cells, thereby allowing expression of the first gene product, wherein the first gene product is a Bax polypeptide, the second gene product is a K2-5F polypeptide, and the protein functionally present in non-cancerous cells but substantially functionally absent in cancerous cells is an SMAD4 protein.

Thus, in some embodiments, the methods, systems, and kits of the presently disclosed subject matter comprise a first nucleic acid encoding a first gene product that is capable of conferring toxicity to the cell, and a second nucleic acid encoding a second gene product that protects non-cancerous cells from developing toxicity by substantially repressing expression of the first gene product and wherein expression of the second gene product is induced by a protein functionally present in non-cancerous cells but substantially functionally absent in cancerous cells, thereby allowing expression of the first gene product, wherein the first gene product is a Puma/Bbc-3 polypeptide, the second gene product is a K2-5F polypeptide, and the protein functionally present in non-cancerous cells but substantially functionally absent in cancerous cells is a p53 protein.

In some embodiments, the methods, systems, and kits of the presently disclosed subject matter comprise a first nucleic acid encoding a first gene product that is capable of conferring toxicity to the cell, and a second nucleic acid encoding a second gene product that protects non-cancerous cells from developing toxicity by substantially repressing expression of the first gene product and wherein expression of the second gene product is induced by a protein functionally present in non-cancerous cells but substantially functionally absent in cancerous cells, thereby allowing expression of the first gene product, wherein the first gene product is a Puma/Bbc-3 polypeptide, the second gene product is a K2-5F polypeptide, and the protein functionally present in non-cancerous cells but substantially functionally absent in cancerous cells is an SMAD4 protein.

Thus, in some embodiments, the methods, systems, and kits of the presently disclosed subject matter comprise a first nucleic acid encoding a first gene product that is capable of conferring toxicity to the cell, and a second nucleic acid encoding a second gene product that protects non-cancerous cells from developing toxicity by substantially repressing expression of the first gene product and wherein expression of the second gene product is induced by a protein functionally present in non-cancerous cells but substantially functionally absent in cancerous cells, thereby allowing expression of the first gene product, wherein the first gene product is an Noxa polypeptide, the second gene product is a K2-5F polypeptide, and the protein functionally present in non-cancerous cells but substantially functionally absent in cancerous cells is a p53 protein.

In some embodiments, the methods, systems, and kits of the presently disclosed subject matter comprise a first nucleic acid encoding a first gene product that is capable of conferring toxicity to the cell, and a second nucleic acid encoding a second gene product that protects non-cancerous cells from developing toxicity by substantially repressing expression of the first gene product and wherein expression of the second gene product is induced by a protein functionally present in non-cancerous cells but substantially functionally absent in cancerous cells, thereby allowing expression of the first gene product, wherein the first gene product is an Noxa polypeptide, the second gene product is a K2-5F polypeptide, and the protein functionally present in non-cancerous cells but substantially functionally absent in cancerous cells is an SMAD4 protein.

In some embodiments, the methods, systems, and kits of the presently disclosed subject matter comprise a first nucleic acid encoding a first gene product that is capable of conferring toxicity to the cell, and a second nucleic acid encoding a second gene product that protects non-cancerous cells from developing toxicity by substantially repressing expression of the first gene product and wherein expression of the second gene product is induced by a protein functionally present in non-cancerous cells but substantially functionally absent in cancerous cells, thereby allowing expression of the first nucleic acid substantially only in cancerous cells, wherein the toxicity is a chemosensitivity, and in some embodiments further comprising contacting the cell with a chemical agent that interacts with the first gene product, wherein an interaction between the chemical agent and the first gene product produces a converted chemical compound that is toxic to the cell, thereby selectively killing cancerous cells having chemosensitivity to the chemical agent, and further wherein the first gene product is an HSV-TK polypeptide and the chemical agent is ganciclovir, the second gene product is a K2-5F polypeptide, the protein functionally present in non-cancerous cells but substantially functionally absent in cancerous cells is an SMAD4 protein.

In some embodiments, the methods, systems, and kits of the presently disclosed subject matter comprise a first nucleic acid encoding a first gene product that is capable of conferring toxicity to the cell, and a second nucleic acid encoding a second gene product that protects non-cancerous cells from developing toxicity by substantially repressing expression of the first gene product and wherein expression of the second gene product is induced by a protein functionally present in non-cancerous cells but substantially functionally absent in cancerous cells, thereby allowing expression of the first gene product, wherein the toxicity is a chemosensitivity, and in some embodiments further comprising contacting the cell with a chemical agent that interacts with the first gene product, wherein an interaction between the chemical agent and the first gene product results in a converted chemical compound that is toxic to the cell, thereby selectively killing cancerous cells having chemosensitivity to the chemical agent, and further wherein the first gene product is an HSV-TK polypeptide and the chemical agent is ganciclovir, the second gene product is a K2-5F polypeptide, the protein functionally present in non-cancerous cells but substantially functionally absent in cancerous cells is a p53 protein.

Thus, in some embodiments, p53 status and the strong repressor activity of designed repressor K2-5F were utilized in the methods, systems, and kits of the presently disclosed subject matter to establish preferential killing of p53-negative cells. The expression of K2-5F was induced by p53 in normal cells, and the K2-5F repressor then inhibited the expression of herpes simplex virus thymidine kinase (HSV-TK) driven by an MDR1 minipromoter. In p53-deficient cells, little K2-5F was expressed, and thus HSV-TK was expressed, allowing the cells to be killed by ganciclovir (GCV). K2-5F induced by exogenous p53 reduced the expression of HSV-TK in human embryonic kidney (HEK) 293 cells, and it subsequently increased cell survival in response to GCV.

To further evaluate this approach in a uniform genetic background, Saos-2 cells stably expressing physiological levels of p53 were paired with wild-type p53-negative Saos-2 cells. Stable expression of moderate levels of p53 in Saos-2 cells induced the expression of K2-5F and reduced HSV-TK expression, resulting in a modest but distinct protection from GCV toxicity. See the Examples below for more detail.

The first nucleic acid and the second nucleic acid of any of the above embodiments of the presently disclosed subject matter can be packaged in plasmids or vectors for use in cells. As used herein, the terms “plasmid” and “vector” are used interchangeably to refer to a small, independently replicating piece of extrachromosomal cytoplasmic DNA that can be transferred from one organism to another. Plasmids, or vectors, are known to persons of ordinary skill in the art and are commonly used for manipulation of nucleic acids.

In some embodiments, the first nucleic acid is present within a first vector and the second nucleic acid is present within a second vector. In some embodiments, the first nucleic acid and the second nucleic acid are present within the same vector. In some embodiments of the presently disclosed subject matter, one or both vectors is selected from the group consisting of an adenovirus vector, a lentivirus vector, a herpes virus vector, a retrovirus vector and an adeno-associated virus vector. In some embodiments, the first nucleic acid is present within a first adenovirus, lentivirus, herpes virus, retrovirus, or adeno-associated virus vector and the second nucleic acid is present within a second adenovirus, lentivirus, herpes virus, retrovirus, or adeno-associated virus vector. In some embodiments, the first nucleic acid and the second nucleic acid are present within the same adenovirus, lentivirus, herpes virus, retrovirus, or adeno-associated virus vector.

The presently disclosed subject matter is suitable for use with any gene delivery vehicle, including viral and nonviral gene delivery vehicles. In some embodiments of the presently disclosed subject matter, the first nucleic acid and the second nucleic acid are packaged in plasmids or vectors, as described above, that are suitable for use in combination with nonviral gene delivery vehicles such as liposomes or nanoparticles for delivery of the nucleic acids to cells.

Nonviral DNA delivery vehicles include polyamines and neutral polymers (synthetic or nonsynthetic, such as gelatin) capable of condensing DNA to nanoparticles with radii of 20-100 nm. Nanoparticles have great potential in providing sustained gene expression in cells and in providing simple and reproducible production, allowing for future up-scaling and commercial production. Thus, nanoparticles are suitable for use in combination with the nucleic acids of the presently disclosed subject matter. See generally Vijayanathan et al., 2002; Brannon-Peppas & Blanchette, 2004.

Nonviral DNA delivery vehicles also include liposomes. Modern drug encapsulation methods allow efficient packing of therapeutic substances inside liposomes, thereby reducing the systemic toxicity of the drugs. Specific targeting can enhance the therapeutic effect of the drugs through their accumulation at the diseased site. Thus, liposomes are suitable for use in combination with the nucleic acids of the presently disclosed subject matter, and can serve to enhance the cancer-killing selectivity of the presently disclosed subject matter. See Felnerova et al., 2004.

III. Viral Vectors

Viral vectors have been widely used as systems for delivering transgenes to cells. Retrovirus, lentivirus, herpes virus, and parvovirus vectors have all been used, and each system has its advantages. Retroviruses have the advantage that they efficiently insert themselves into a host chromosome, ensuring long-term expression. “Pseudotyped” variants of retrovirus are available that will insert themselves into all cells or only into specific cells. However, the efficient insertion of retroviruses into host chromosomes is also a disadvantage, as random insertions can create mutations that are deleterious. Also, some retroviruses require that the host cell be in a state of replication before they can integrate into the host genome.

Lentiviruses, including human immunodeficiency virus (HIV), are a group of related retroviruses. Like other retrovirus vectors, lentivirus vectors can accommodate transgenes up to about 8 kb in length, and can be prevented from replicating by eliminating essential viral genes. Unlike other retrovirus vectors, lentivirus vectors are able to infect nondividing cells.

Herpes simplex virus is a double-stranded DNA virus with a 152-kb genome. Its relatively large size means that it could be used for manipulation of larger transgenes and even multiple transgenes. Herpes simplex virus can infect a wide variety of cell types in both the dividing and the nondividing state.

Retrovirus, lentivirus, and herpes virus vectors are suitable for use in the methods, systems, and kits of the presently disclosed subject matter, and these vectors are particularly useful in the field of gene therapy, as reviewed in Lundstrom, 2004.

Parvoviruses, including adenoviruses, are small, single-stranded, non-enveloped DNA viruses between twenty to thirty nanometers in diameter. The genomes of parvoviruses are approximately 5000 nucleotides long, containing two open reading frames. The left-hand open reading frame encodes the proteins responsible for replication (Rep), while the right-hand open reading frame encodes the structural proteins of the capsid (Cap). All parvoviruses have virions with icosahedral symmetry composed of a major Cap protein, usually the smallest of the Cap proteins, and one or two minor Cap proteins. The Cap proteins are generated from a single gene that initiates translation from different start codons. These proteins have identical C-termini, but possess unique N-termini due to different initiation codons.

Most parvoviruses have narrow host ranges; the tropism of B19 is for human erythroid cells (Munshi et al., (1993) J. Virology 67:562), while canine parvovirus has a tropism for lymphocytes in adult dogs (Parrish et al., (1988) Virology 166:293; Chang et al., (1992) J. Virology 66:6858). Adeno-associated virus (MV), on the other hand, can replicate well in canine, mouse, chicken, bovine, monkey cells, as well as numerous human cells and cell lines, when the appropriate helper virus is present. In the absence of helper virus, MV will infect and establish latency in all of these cell types, suggesting that the MV receptor is common and conserved among species. Several serotypes of MV have been identified, including serotypes 1, 2, 3, 4, 5 and 6.

Adeno-associated virus (MV) is a dependent parvovirus twenty nanometers in size which requires co-infection with another virus (either adenovirus or certain members of the herpes virus group) to undergo a productive infection in cells. In the absence of co-infection with helper virus, the MV virion binds to a cellular receptor and enters the cell, migrates to the nucleus, and delivers a single-stranded DNA genome that can establish latency by integration into the host chromosome. The interest in MV as a vector has centered around the biology of this virus. In addition to its unique life cycle, MV has a broad host range for infectivity (human, mouse, monkey, dog, etc.), is ubiquitous in humans, and is completely nonpathogenic.

The finite packaging capacity of this virus (4.5 kb) has restricted the use of this vector in the past to small genes or cDNAs. To advance the prospects of MV gene delivery, vectors sufficient to carry larger genes must be developed. In addition, virions that specifically and efficiently target defined cell types without transducing others are beneficial for clinical applications.

Parvovirus and MV vectors are suitable for use in the methods, systems, and kits of the presently disclosed subject matter, and these vectors are particularly useful in the field of gene therapy. Representative vectors that can be employed in the methods, systems, and kits of the presently disclosed subject matter are described in U.S. Pat. No. 6,458,587; U.S. Pat. No. 6,489,162; U.S. Pat. No. 6,491,907; and U.S. Pat. No. 6,548,286, the contents of which are incorporated in their entireties by reference.

In some embodiments, an adenovirus vector of the presently disclosed subject matter is conditionally replication competent. That is, it contains one or more functional genes required for its replication placed under the transcriptional control of an inducible promoter. This inhibits uncontrolled replication in vivo and reduces undesirable side effects of viral infection. Replication competent self-limiting or self-destructing viral vectors can also be used, as can replication deficient viral vectors.

Incorporation of a nucleic acid construct into a viral genome can be optionally performed by ligating the construct into an appropriate restriction site in the genome of the virus. Viral genomes can then be packaged into viral coats or capsids by any suitable procedure. In particular, any suitable packaging cell line can be used to generate viral vectors of the presently disclosed subject matter. These packaging lines complement the conditionally replication deficient viral genomes of the presently disclosed subject matter, as they include, typically incorporated into their genomes, the genes which have been put under an inducible promoter deleted in the conditionally replication competent vectors. Thus, the use of packaging lines allows viral vectors of the presently disclosed subject matter to be generated in culture.

IV. Transgenes

In order to efficiently and selectively kill a cancerous cell, the presently disclosed subject matter provides in some embodiments nucleic acids comprising a transgene. In accordance with the presently disclosed subject matter, a transgene can comprise a repressor gene, an apoptosis-inducing gene, a suicide prodrug-converting enzyme gene, a bacterial toxin gene, an antisense gene, an immunostimulatory gene, or combinations thereof.

As used herein, the term “transgene” refers to any nucleotide sequence to be introduced into a cell, thereby allowing the nucleotide sequence to be expressed in the cell. A transgene can include a nucleotide sequence that is partly or entirely heterologous (i.e. foreign) to the organism from which the cell was derived, or can be a nucleotide sequence identical or homologous to a nucleotide sequence already present within the cell.

The methods of the presently disclosed subject matter can include a transgene comprising a nucleic acid sequence that encodes a polypeptide having a therapeutic biological activity (also referred to herein as a “therapeutic polypeptide”). Exemplary therapeutic polypeptides include but are not limited to immunostimulatory molecules, suicide gene products, repressor gene products, bacterial toxin gene products, and anti-angiogenic factors (see Mackensen et al., 1997; Walther & Stein, 1999; Kirk & Mule, 2000 and references cited therein). In some embodiments of the presently disclosed subject matter, a transgene comprises a nucleic acid sequence that encodes a therapeutic polypeptide. In some embodiments, a transgene comprises a nucleic acid sequence that encodes a polypeptide having a repressor effect on the functionality of a suicide gene. In some embodiments, a transgene comprises a nucleic acid sequence that encodes a suicide gene. In some embodiments, a transgene comprises a nucleic acid sequence that encodes a gene product that is directly toxic to the cell, such as a bacterial toxin. It is clear to those of skill in the art that a single cell can comprise more than one transgene. Thus, in some embodiments of the presently disclosed subject matter, a first transgene comprises a nucleic acid sequence that encodes a suicide gene and a second transgene comprising a nucleic acid sequence encodes a polypeptide having a repressor effect on the functionality of the suicide gene.

As used herein, the term “suicide gene” refers to a coding sequence that encodes a polypeptide, the product of which causes the cell to die. A suicide gene can encode a gene that causes cell death directly, for example by inducing apoptosis. Such a gene is referred to as an “apoptosis-inducing gene”, and includes, but is not limited to TNF-α (Idriss & Naismith, 2000), Trail (Srivastava, 2001), Bax, Bad, and Bcl-2 (Shen & White, 2001). Other genes that encode proteins that kill cells directly include bacterial toxin genes, which are normally found in the genome of certain bacteria and encode polypeptides (i.e. bacterial toxins) that are toxic to eukaryotic cells. Bacterial toxins include, but are not limited to diphtheria toxin (Frankel et al., 2001).

Additionally, a suicide gene can encode a polypeptide that converts a prodrug to a toxic compound. Such suicide prodrug-converting enzymes include, but are not limited to the HSV-TK polypeptide, which converts ganciclovir to a toxic nucleotide analog (Freeman et al., 1996); cytosine deaminase, which converts the non-toxic nucleotide analog 5-fluorocytosine into the toxic analog 5-fluorouracil (Yazawa et al., 2002); and cytochrome p450, which converts certain aliphatic amine N-oxides into toxic metabolites (Patterson, 2002).

Additionally, a suicide gene can encode a polypeptide that interferes with a signal transduction cascade involved with cellular survival or proliferation. Such cascades include, but are not limited to, the cascades mediated by the Flt1 and Flk1 receptor tyrosine kinases (reviewed in Klohs, et al., 1997). Polypeptides that can interfere with Flt1 and/or Flk1 signal transduction include, but are not limited to, a soluble Flt1 receptor (s-Flt1; Shibuya, 2001) and an extracellular domain of the Flk-1 receptor (ex-Flk1; Lin et al., 1998).

V. Therapy Methods

A therapeutic method according to the presently disclosed subject matter comprises in some embodiments contacting a cancerous cell with a vector, whereby the vector enters the cell and inhibits tumor growth. For example, the disclosed methods, systems, and kits can be useful in the treatment of both primary and metastatic solid tumors and carcinomas of the breast; colon; rectum; lung; oropharynx; hypopharynx; esophagus; stomach; pancreas; liver; gallbladder; bile ducts; small intestine; urinary tract including kidney, bladder and urothelium; female genital tract including cervix, uterus, ovaries, choriocarcinoma and gestational trophoblastic disease; male genital tract including prostate, seminal vesicles, testes and germ cell tumors; endocrine glands including thyroid, adrenal, and pituitary; skin including hemangiomas, melanomas, sarcomas arising from bone or soft tissues and Kaposi's sarcoma; tumors of the brain, nerves, eyes, and meninges including astrocytomas, gliomas, glioblastomas, retinoblastomas, neuromas, neuroblastomas, Schwannomas and meningiomas; solid tumors arising from hematopoietic malignancies such as leukemias and including chloromas, plasmacytomas, plaques and tumors of mycosis fungoides and cutaneous T-cell lymphoma/leukemia; lymphomas including both Hodgkin's and non-Hodgkin's lymphomas.

The methods, systems, and kits of the presently disclosed subject matter can also be employed for the prevention of metastases from the tumors described above either when used alone or in combination with radiotherapeutic, photodynamic, and/or chemotherapeutic treatments conventionally administered to patients for treating disorders, including angiogenic disorders. For example, a tumor can be treated conventionally with surgery, photodynamic therapy, radiation and/or chemotherapy followed by administration of the methods, systems, and kits of the presently disclosed subject matter to extend the dormancy of micrometastases and to stabilize and inhibit the growth of any residual primary tumor. Indeed, administration of the methods, systems, and kits of the present disclosed subject matter can be provided before, during, or after radiotherapy; before, during, or after chemotherapy; and/or before, during, or after photodynamic therapy.

The methods, systems, and kits of the presently disclosed subject matter can be used in treatment of any cancer in which the cancerous cells lack the functional presence of a tumor suppressor that can be operably linked to a repressor according to the methods, systems, and kits of the presently disclosed subject matter.

In some embodiments of the presently disclosed subject matter, methods are provided for selective killing of a cancerous cell by providing to the cell two different vectors, a first vector comprising a repressor gene under the transcriptional regulation of a tumor suppressor, and a second vector comprising a suicide gene operably linked to a binding site for the gene product of the repressor gene on the first vector. The use of a combination approach offers advantages in that the use of two vectors in conjunction with one another can expand the ability of certain vectors (e.g. some adenovirus vectors) to deliver transgenes.

In some embodiments of the presently disclosed subject matter, methods are provided for selective killing of a cancerous cell by providing to the cell a single vector comprising both a repressor gene under the transcription regulation of a tumor suppressor and a suicide gene operably linked to a binding site for the gene product of the repressor gene. The single vector approach is accomplished by reducing the overall size of the nucleic acid construct. One way of accomplishing this is by reducing the size of one or more transcription regulatory elements, such as a promoter. See Example 15 below for more detail.

V.A. Subjects

The subject treated in the presently disclosed subject matter in its many embodiments is desirably a human subject, although it is to be understood that the principles of the presently disclosed subject matter indicate that the presently disclosed subject matter is effective with respect to invertebrate and to all vertebrate species, including mammals, which are intended to be included in the term “subject”. Moreover, a mammal is understood to include any mammalian species in which treatment or prevention of cancer is desirable, particularly agricultural and domestic mammalian species.

The methods of the presently disclosed subject matter are particularly useful in the treatment of warm-blooded vertebrates. Thus, the presently disclosed subject matter concerns mammals and birds.

More particularly provided is the treatment of mammals such as humans, as well as those mammals of importance due to being endangered (such as Siberian tigers), of economic importance (animals raised on farms for consumption by humans) and/or social importance (animals kept as pets or in zoos, or animals used in laboratory research, such as rodents and non-human primates) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, i.e., poultry, such as turkeys, chickens, ducks, geese, guinea fowl, and the like, as they are also of economic importance to humans. Thus, contemplated is the treatment of livestock, including, but not limited to, domesticated swine (pigs and hogs), ruminants, horses, poultry, and the like.

The subject treated in the presently disclosed subject matter in some embodiments can also be an immortalized cell line, for example, a cell line of mammalian origin. Many cell lines are appropriate for use with the methods, systems, and kits of the presently disclosed subject matter, including but not limited to HEK 293T cells, Saos-2 cells, HepG2 cells, Hep-2 cells, Panc-1 cells, AsPc-1 cells, SF-539 cells, SKNMC cells, IMR-32 cells, SKNSH cells, SNB-78 cells, Colo-205 cells, HCT-15 cells, HT-29 cells, SW-620 cells, A-549 cells, NCl-H23 cells, HOP-18 cells, KB human epidermal carcinoma cells, OVCAR-5 cells, NIH-OVCAR-3 cells, SK-OV-3 cells, DU-145 cells, PC-3 cells, MCF-7 cells, T-47-D cells, BT-483, BT-474, Hs-578T, ZR-75, MD-MB-468, MD-MB-435, BT-20, Hela cells, and SiHa cells.

V.B. Formulation

The nucleic acids of the presently disclosed subject matter comprise in some embodiments a composition that comprises a pharmaceutically or physiologically acceptable carrier, for example a carrier that is pharmaceutically acceptable in humans. Any suitable pharmaceutical formulation can be used to prepare the nucleic acids for administration to a subject.

For example, suitable formulations can include aqueous and non-aqueous sterile injection solutions which can contain anti-oxidants, buffers, bacteriostats, bactericidal antibiotics and solutes which render the formulation isotonic with the bodily fluids of the intended recipient; and aqueous and non-aqueous sterile suspensions which can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients are SDS, in the range of about 0.1 to about 10 mg/ml, in some embodiments about 2.0 mg/ml; and/or mannitol or another sugar, for example in the range of about 10 to about 100 mg/ml, in some embodiments about 30 mg/ml; and/or phosphate-buffered saline (PBS).

It should be understood that in addition to the ingredients particularly mentioned above, the formulations of the presently disclosed subject matter can include other agents conventional in the art having regard to the type of formulation in question. Of the possible formulations, sterile pyrogen-free aqueous and non-aqueous solutions can be used.

The therapeutic regimens and pharmaceutical compositions of the presently disclosed subject matter can also be combined with other treatments for cancer to enhance therapeutic effects. Examples of such other treatments include any treatment involving the administration of drugs that damage DNA. Exemplary DNA damaging drugs include, but are not limited to, alkylating agents such as nitrogen mustards, intercalators like doxorubicin, and topoisomerase inhibitors like etoposide. All of the aforementioned drugs elevate p53 levels in normal cells and thus potentially increase the ability of the presently disclosed subject matter to distinguish between p53-positive healthy cells and p53-negative cancerous cells, in terms of p53-mediated expression of the designed repressor.

V.C. Administration

Suitable methods for administration of the nucleic acids of the presently disclosed subject matter include, but are not limited to intravenous or intratumoral injection. Alternatively, the nucleic acids of the presently disclosed subject matter can be deposited at a site in need of treatment in any other manner, for example by spraying a composition comprising the nucleic acids within the pulmonary pathways. The particular mode of administering a therapeutic composition of the presently disclosed subject matter depends on various factors, including the locations, distribution and abundance of cells to be treated, the vector employed, additional tissue- or cell-targeting features of the nucleic acids, and mechanisms for metabolism or removal of the nucleic acids from the site of administration. For example, relatively superficial tumors can be injected intratumorally. By contrast, internal tumors can be treated by intravenous injection.

In some embodiments, the method of administration encompasses features for regionalized nucleic acid delivery or accumulation at the site in need of treatment. In some embodiments, the nucleic acids are delivered intratumorally. In some embodiments, selective delivery of the nucleic acids to a tumor is accomplished by intravenous injection.

For delivery of the nucleic acids to pulmonary pathways, the nucleic acids of the presently disclosed subject matter can be formulated as an aerosol or coarse spray. Methods for preparation and administration of aerosol or spray formulations can be found, for example, in Cipolla et al., 2000 and in U.S. Pat. Nos. 5,858,784; 6,013,638; 6,022,737; and 6,136,295. It is noted that in the methods, systems, and kits of the presently disclosed subject matter, physical delivery of the therapeutic composition directly to the cancerous cells is not a necessity, as an inherent selectivity for cancerous cells is a feature the presently disclosed subject matter and thus removes the need to apply the therapy specifically and directly to cancerous cells. However, delivery of the therapy directly to the cancerous cells can be done when feasible.

V.D. Dose

An effective dose of the nucleic acids of the presently disclosed subject matter is administered to a subject in need thereof. A “therapeutically effective amount” is an amount of the therapeutic composition sufficient to produce a measurable response (e.g., a cytolytic response in a subject being treated). In some embodiments, an activity that inhibits tumor growth is measured. Actual dosage levels of active ingredients in the pharmaceutical compositions of the presently disclosed subject matter can be varied so as to administer an amount of the active compound(s) that is effective to achieve the desired therapeutic response for a particular subject. The selected dosage level will depend upon the activity of the therapeutic composition, the route of administration, combination with other drugs or treatments, the severity of the condition being treated, and the condition and prior medical history of the subject being treated. However, it is within the skill of the art to start doses of the compound at levels lower than required to achieve the desired therapeutic effect and to gradually increase the dosage until the desired effect is achieved.

The potency of a therapeutic composition can vary, and therefore a “therapeutically effective” amount can vary. However, one skilled in the art can readily assess the potency and efficacy of a composition of the presently disclosed subject matter and adjust the therapeutic regimen accordingly.

After review of the disclosure herein of the presently disclosed subject matter, one of ordinary skill in the art can tailor the dosages to an individual patient, taking into account the particular formulation, method of administration to be used with the composition, and tumor size. Further calculations of dose can consider patient height and weight, severity and stage of symptoms, and the presence of additional deleterious physical conditions. Such adjustments or variations, as well as evaluation of when and how to make such adjustments or variations, are well known to those of ordinary skill in the art of medicine.

In some embodiments, the nucleic acids of the presently disclosed subject matter are packaged in a viral vector. For local administration of viral vectors, previous clinical studies have demonstrated that up to 10¹³ plaque forming units (pfu) of virus can be injected with minimal toxicity. In human patients, 1×10⁹-1×10¹³ pfu are routinely used (see Habib et al., 1999). To determine an appropriate dose within this range, preliminary treatments can begin with 1×10⁹ pfu, and the dose level can be escalated in the absence of dose-limiting toxicity. Toxicity can be assessed using criteria set forth by the National Cancer Institute and is reasonably defined as any grade 4 toxicity or any grade 3 toxicity persisting more than 1 week. Dose is also modified to maximize anti-tumor or anti-angiogenic activity. Representative criteria and methods for assessing anti-tumor and/or anti-angiogenic activity are described herein below. With replicative virus vectors, a dosage of about 1×10⁷ to 1×10⁸ pfu can be used in some instances.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

### Example 1

### Cell Culture

U-2OS and Saos-2 human osteosarcoma cancer cell lines, as well as HEK 293 and HEK 293T cells, were purchased from the Tissue Culture Facility (Lineberger Comprehensive Cancer Center, University of North Carolina at Chapel Hill, Chapel Hill, N.C., United States of America). Saos-2 and U-2OS cells were cultured in McCoy's 5a medium (Invitrogen, Carlsbad, Calif., United States of America) supplemented with 10% fetal bovine serum. Both HEK 293T and HEK 293 cells were grown in Dulbecco's modified Eagle's medium (Invitrogen, Carlsbad, Calif., United States of America) with 10% fetal bovine serum.

### Example 2

### Plasmid Construction

Two copies of the K2-5F binding sequence in the MDR1 promoter were excised with BamHI from plasmid 2MDR-LUC (Bartsevich & Juliano, 2000) and then cloned into Bg/II-digested pRL-TK (Promega Corp., Madison, Wis., United States of America) to create pRL-2MDR-TKp. A full-length cDNA of HSV-TK with NheI and NotI sites at the ends and including a polyhistidine tag at the carboxyl terminus was obtained by thirty rounds of PCR amplification of plasmid LNC-TK (Hoganson et al., 1996). The oligonucleotide primers used were 5′-TAGGCTAGCCACCATGGCTTCGTACCCCTGCCA-3′ (SEQ ID NO: 1) and 5′-GAAGCGGCCGCTCTAGMTCMTGATGATGATGATGATGGTTAG CCTCCCCCATCT-3′ (SEQ ID NO: 2). The amplified cDNAs were inserted into NheI- and NotI-digested pRL-2MDR-TKp and pRL-TK, resulting in vectors pRL-2MDR-TKp-TK (producing HSV-TK regulated by K2-5F) and pRL-TKp-TK (producing HSV-TK not regulated by K2-5F), respectively. The recombinant molecules were sequenced to verify that no mutations had been introduced during PCR amplification and cloning.

The p53-responsive vector pFR-2p21-K2-5F is based on the reporter plasmid pFR*-2p21 (Falke et al., 2003), which contains two copies of the p53 binding site of the p21 promoter. The luciferase gene of pFR*-2p21 was excised with EcoRI/XbaI and replaced by an EcoRI/SpeI-digested linker containing the restriction sites KpnI, NcoI, and XbaI. K2-5F sequence with NcoI and XbaI sites at the ends and including a myc tag at the carboxyl terminus was obtained by PCR amplification of plasmid pcK2-5F (Bartsevich & Juliano, 2000) and inserted into the NcoI/XbaI-digested pFR*-plasmid, resulting in plasmid pFR-2p21-K2-5F. Primers used to create the linker were 5′-GCGAATTCCAGCTTGGCATTCCGGTACTGTTGGTACCATGGCGTCTAG AC-3′ (SEQ ID NO: 3) and 5′-GCACTAGTGTATTACMTAGCTAAGMTTTCGTCTAGACGCCATGGTAC C-3′ (SEQ ID NO: 4). The primers were annealed, and the ends were filled in with T4 DNA polymerase. The primers used to amplify K2-5F were 5′-CCACCATGGCTAGCTGTTTC-3′ (SEQ ID NO: 5) and 5′ CGTCTAGACTGMTACAGTTACATTTCMTGATGATGATGATGAT-3′ (SEQ ID NO: 6).

Plasmid pCMV-P53-Myc was constructed from vector pcDNA3.1(−)/Myc-HisA (Stratagene, La Jolla, Calif., United States of America) by inserting a p53 coding sequence from plasmid pCMVP53 (BD Biosciences Clontech, Palo Alto, Calif., United States of America) through HindIII/EcoRI sites.

### Example 3

### Transfection

Transfection was carried out using FuGENE 6 (Roche Diagnostics, Indianapolis, Ind., United States of America) according to the manufacturer's instructions. Cells were co-transfected with the indicated vectors. The total amount of DNA was adjusted with empty vectors or salmon sperm DNA.

### Example 4

### Immunoprecipitation, Nickel-Bead Purification, and Western Blotting

Forty eight hours after transfection, cells were lysed in modified RIPA buffer (50 mM Tris-HCl, pH 7.5, 150 mM NaCl, 1% Nonidet P40, 0.5% deoxycholate, 2 mM phenylmethylsulfonyl fluoride, and 0.1% aprotinin), and lysates were centrifuged at 12,000 rpm for ten minutes at 4° C. For Western blotting, equal amounts of protein (20 μg) were mixed with 2×SDS sample buffer and boiled for five minutes. For nickel-bead captures, the supernatants were incubated with aprotinin-pretreated nickel beads for two hours. The captured beads were washed three times with the modified RIPA buffer and boiled with 1×SDS sample buffer (with the addition of 200 mM imidazole) for five minutes.

For immunoprecipitation, the supernatants were incubated with antibody for two hours at 4° C. followed by the addition of protein G-sepharose™ and further incubation for two hours at 4° C. The precipitates were washed three times with modified RIPA buffer and boiled with 1× SDS sample buffer for five minutes. The proteins were subjected to 10% SDS-polyacrylamide gel electrophoresis, and the separated proteins were transferred onto polyvinylidene difluoride membranes (Millipore Corporation, Bedford, Mass., United States of America). K2-5F was detected using monoclonal anti-c-myc antibody 9E10 (Berkeley Antibody Company, Richmond, Calif., United States of America) at a dilution of 1:2000. HSV-TK was detected using monoclonal anti-polyhistine antibody H-3 (Santa Cruz Biotechnology, Inc., Santa Cruz, Calif., United States of America) at a dilution of 1:1000. p53 was detected using monoclonal anti-p53 antibody DO-1 (Santa Cruz Biotechnology, Inc.) at a dilution of 1:5000. Secondary antibody was peroxidase conjugated goat anti-mouse IgG antibody (Calbiochem, San Diego, Calif., United States of America) at a dilution of 1:5000. Immunoprecipitated K2-5F and p53 were detected using biotin-labeled anti-c-myc antibody 9E10 (Berkeley Antibody Company) at a dilution of 1:1000 followed by streptavidin-horseradish peroxidase at a dilution of 1:5000 (Amersham Biosciences Inc., Arlington Heights, Ill., United States of America). Signals were detected by enhanced chemiluminescence (ECL kit, Amersham Biosciences Inc.).

### Example 5

### GCV Sensitivity Assays

Cells were seeded in twelve-well plates and transfected as described above. The transfection mixture also included luciferase-expressing plasmid pGL3 (Promega) or β-galactosidase-expressing plasmid to mark the transfected cells. Twenty-four hours after transfection, cells in each well were divided equally into six parts and re-plated into six-well plates. Twenty-four hours later, GCV (Sigma Chemical Co., St. Louis, Mo., United States of America) was administered in varying concentrations (Mavria & Porter, 2001). After four days of treatment, cells were lysed, and luciferase activities or β-galactosidase activities were measured according to the manufacturer's protocol (Promega Corp., Madison, Wis., United States of America). Cell viability was estimated by luciferase activity or β-galactosidase activity, with results expressed as a percentage of the activity in the absence of GCV. Although the transfection of various plasmids may affect total luciferase or β-galactosidase expression, each concentration-response profile is normalized against its own control at zero concentration of GCV; thus, the relative response is independent of differences in the absolute level of enzymatic activity. Therefore, in this assay, the amount of luciferase or β-galactosidase retained after GCV treatment is an indicator of cell survival.

### Example 6

### Stable Cell Line Production

Saos-2 cells were transfected with linearized pCMV-P53-Myc vector or with pcDNA3.1 vector as a control. Forty-eight hours after transfection, cells resistant to neomycin were selected in medium containing 1 mg/ml G418 (Invitrogen, Carlsbad, Calif., United States of America). Clones were tested for constitutive p53 expression by Western blot. Eight p53-positive clones (Saos-2/p53+) were selected and maintained in the presence of G418 (0.5 mg/ml), as were several p53-negative control clones (Saos-2/p53-).

### Example 7

### Colony-Formation Assay

Cells were transfected with the plasmids indicated, along with a β-galactosidase-expressing plasmid to mark the transfected cells. Twenty-four hours after transfection, 600 cells were re-plated in 10-cm plates, and after another twenty four hours, GCV was administered in varying concentrations. After 10 days, surviving blue (β-galactosidase-positive) colonies larger than 50 cells were counted. Survival was expressed as a percentage of colonies formed at 0 GCV.

### Example 8

### Plasmid Construction

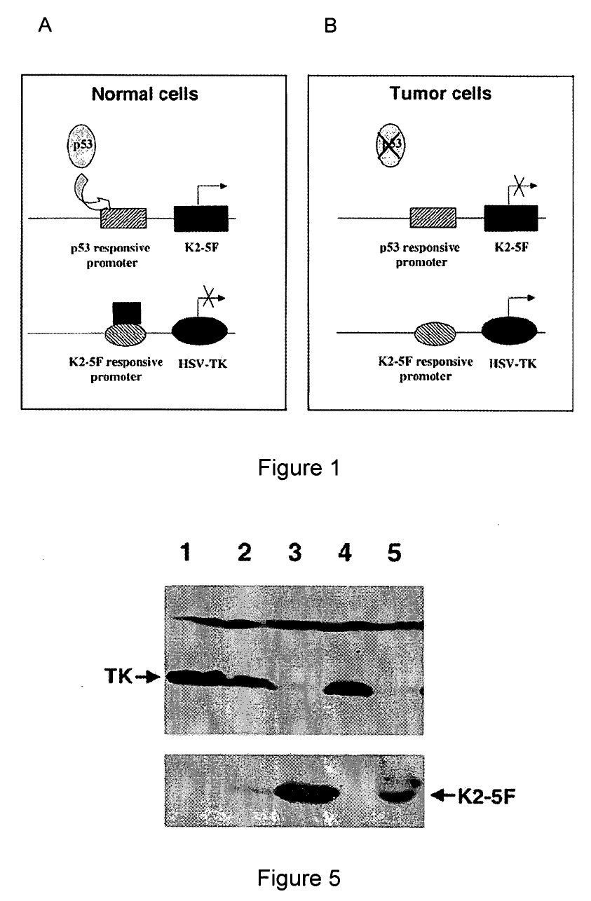

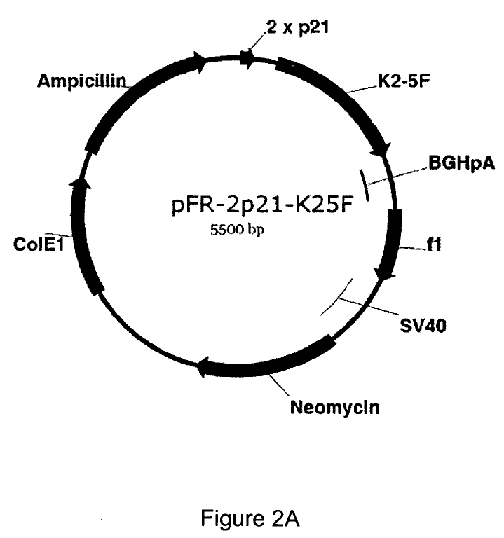

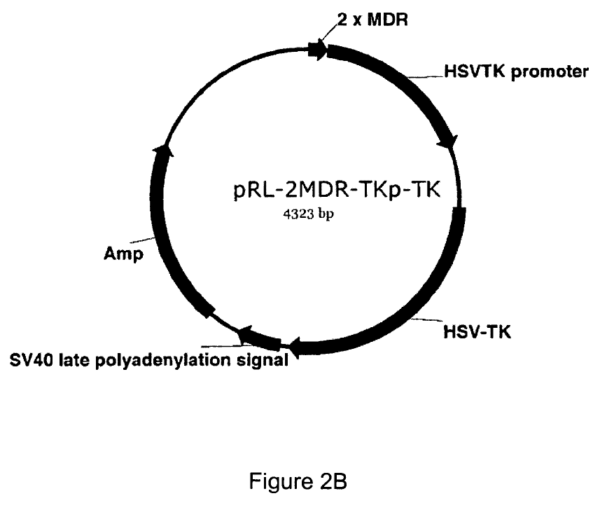

A strategy to selectively kill p53-negative tumor cells is shown in FIG. 1. A set of vectors was developed that permitted effective cell killing in the absence of functional p53, but not in its presence. To attain this, two plasmids were constructed. The first, pFR-2p21-K2-5F (FIG. 2A), contained two copies of a p53 binding sequence taken from the p21 gene, followed by a TATA box and then the coding sequence of the K2-5F-designed repressor protein. This vector expressed K2-5F in the presence of functional p53. The second vector, pRL-2MDR-TKp-TK (FIG. 2B), contained two copies of a 15-base sequence (taken from the MDR1 promoter) that specifically binds K2-5F; the two copies were placed upstream of a partial HSV-TK promoter, which in turn was upstream from the HSV-TK coding sequence. This vector constitutively expressed HSV-TK, but was repressed by the expression of K2-5F. Thus, when both vectors were present in a cell, there were high levels of HSV-TK expression in the absence of p53 and low levels in its presence.

### Example 9

### Repression of HSV-TK by Constitutively Expressed K2-5F

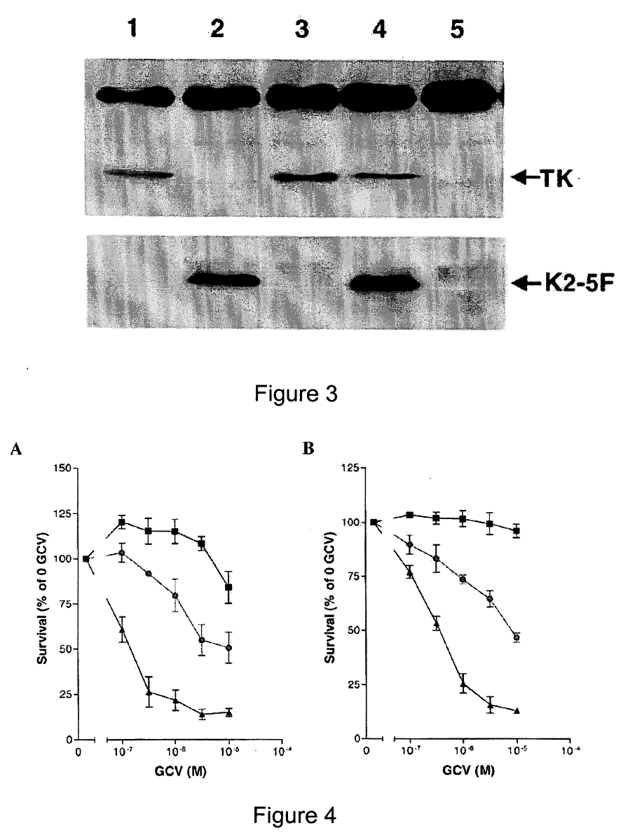

To initially assess the ability of K2-5F to repress the expression of HSV-TK from the pRL-2MDR-TKp-TK vector, a vector was used that constitutively expressed K2-5F driven by a cytomegalovirus promoter. HEK 293T cells were transfected with equal amounts of the plasmids pcK2-5F and pRL-2MDR-TKp-TK (lane 2), pRL-TKp-TK and pcDNA3.1A (lane 3), pcK2-5F and pRL-TKp-TK (lane 4); or with empty vectors pcDNA3.1A and pRL-2MDR-TKp-TK (lane 1) or PRL-TK (lane 5). As seen from the Western blotting result presented in FIG. 3, constitutively expressed K2-5F dramatically inhibited the expression of HSV-TK from pRL-2MDR-TKp-TK, but not from pRL-TKp-TK, suggesting that the repression was attributed to the two copies of the K2-5F-binding 15-base sequence from the MDR1 promoter.

### Example 10

### Regulation of the Response to GCV by Constitutively Expressed K2-5F

HEK 293T cells were co-transfected with pRL-2MDR-TKp-TK and pcK2-5F or empty vector pcDNA3.1A and with the luciferase vector pGL3 (FIG. 4A) or β-galactosidase-expressing vector (FIG. 4B). Cells transfected with two empty vectors (pcDNA3.1A and PRL-TK) were used as controls. After four days of treatment with GCV, luciferase or β-galactosidase activity was measured as an indication of cell viability. The repression of HSV-TK expression by K2-5F resulted in a significant right shift of the dose-response profile of cells co-transfected with pRL-2MDR-TKp-TK and pcK2-5F. Thus, the IC₅₀ for GCV in the cells transfected with pRL-2MDR-TKp-TK alone was approximately 2×10⁻⁷ M, whereas that in the cells transfected with both pRL-2MDR-TKp-TK and pcK2-5F was approximately 1×10⁻⁵M. Therefore, constitutively expressed K2-5F substantially reduced cell killing by HSV-TK and GCV. The use of two reporter genes, luciferase or β-galactosidase, validates the reliability of this assay. In addition, the use of the β-galactosidase marker allowed an estimate of the transfection efficiency, which was approximately 60%. These results showed that the expression of HSV-TK from pRL-2MDR-TKp-TK was substantially inhibited by constitutively expressed K2-5F.

### Example 11

### Repression of HSV-TK by p53-Induced K2-5F

In order to test whether inhibition similar to that seen in Example 10 could be achieved through p53-mediated induction of K2-5F, HEK 293 cells were used, which also have high transfection efficiency but lack the T-antigen, because the T-antigen in HEK 293T cells interacts with and inactivates p53. As represented in FIG. 5, HEK 293 cells were co-transfected with the following plasmids: pRL-2MDR-TKp-TK (lane 1), pRL-2MDR-TKp-TK and pFR-2p21-K2-5F (lane 2), pRL-2MDR-TKp-TK, pFR-2p21-K2-5F and pCMV-P53-Myc (lane 3), pRL-2MDR-TKp-TK and PCMV-P53-Myc (lane 4), or pRL-2MDR-TKp-TK and pcK2-5F (lane 5). FIG. 5 shows that K2-5F was strongly induced by co-transfection with a p53-expressing plasmid. Furthermore, this led to a dramatic p53-dependent reduction in the level of expression of HSV-TK. The small amount of K2-5F seen in the absence of co-transfected p53 might be caused by endogenous p53 in the HEK 293 cells or by some degree of “leakiness” in the promoter for K2-5F expression.

### Example 12

### Regulation of the Response to GCV by p53-Induced K2-5F

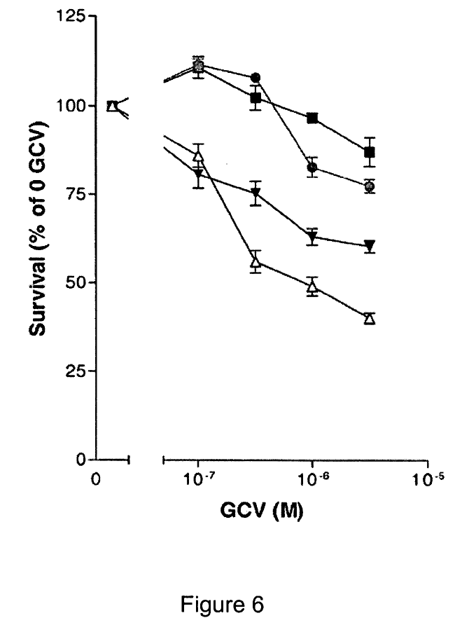

To test the pharmacological consequences of p53-mediated expression of K2-5F, HEK 293 cells were transfected with the luciferase reporter plasmid and with various combinations of TK-, K2-5F-, and p53-expressing plasmids. The cells were then treated with GCV, and cell viability was evaluated by the use of the luciferase assay. As seen in FIG. 6, the inhibition of HSV-TK expression by p53-induced K2-5F resulted in an approximately 1-log right shift of the dose-response profile of cells cotransfected with these three vectors compared with cells transfected with the TK vector only or with the TK vector and the K2-5F vector. Although the rescue of cell viability was not as pronounced as the one caused by constitutively expressed K2-5F, the impact of p53-induced K2-5F was very clear. Because p53 itself is toxic to some degree, only low levels of p53 expression were used.

### Example 13

### Repression of HSV-TK by p53-Induced K2-5F in Saos-2 Cells

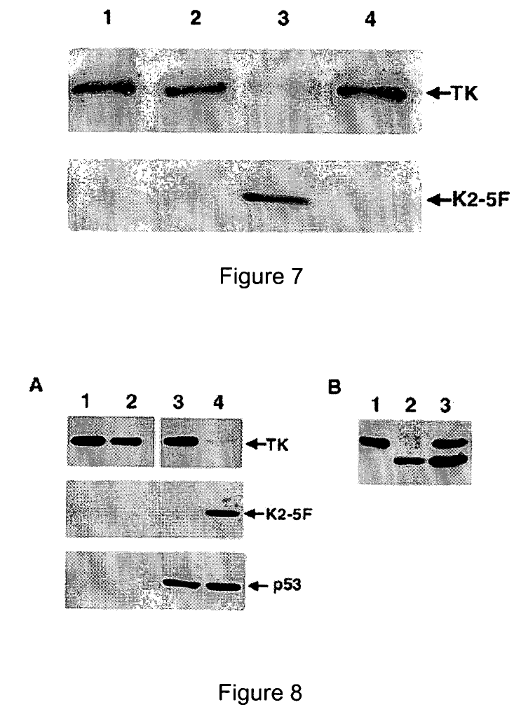

Saos-2 cells, a p53-null osteosarcoma cell line, were used to test whether p53-deficient tumor cells would be killed by expression of a suicide gene. First, it was determined whether TK expression from pRL-2MDR-TKp-TK could be repressed by p53-induced K2-5F in these cells. Cells were cotransfected with various combinations of TK-, K2-5F-, and p53-expressing plasmids. Since protein expression is much lower in Saos-2 cells than in HEK 293 cells, hexahistidine and myctagged-expressed proteins were enriched by nickel-bead affinity or by immmunoprecipitation with anti-myc antibody. FIG. 7 shows that p53-induced K2-5F effectively repressed the expression of the HSV-TK suicide gene in SAOS-2 cells.

Pairs of cell lines were established that were derived from the same genetic background but with different p53 status. Saos-2 cells were transfected with pCMV-P53-Myc or pcDNA3.1 and stably transfected cell lines were selected with G418. Saos-2 cells stably transfected with pcDNA3.1 served as p53-negative controls (designated as Saos-2/p53-). Several clones stably transfected with pCMV-P53-Myc expressed p53, as detected by Western blotting; these clones were designated as Saos-2/p53+. Both Saos-2/p53- and Saos-2/p53+ cells were cotransfected with a TK-expressing plasmid along with empty vector or K2-5F-expressing plasmid. As shown in FIG. 8A, the chromosome-integrated endogenous p53 induced the expression of K2-5F, which then inhibited HSV-TK expression from pRL-2MDR-TKp-TK. The p53 in the Saos-2/p53+ clone was considered to be at a physiological level because it approximated the p53 level in U-20S cells, a p53 wild-type osteosarcoma cell line that is similar to SAOS-2 (FIG. 8B).

### Example 14

### Regulation of the Response to GCV by Endogenous p53-Induced K2-5F

The pharmacological effect of K2-5F induced by endogenous p53 in stably transfected Saos-2 cells was investigated. Saos-2/p53- and Saos-2/p53+ cells were co-transfected with TK-expressing plasmid and K2-5F-expressing plasmid or empty vector. The cells were then treated with GCV, and cell viability was evaluated with the use of a colony-formation assay. This assay was chosen so as to validate the pharmacological response using an alternative to the biochemical reporter assays described above.

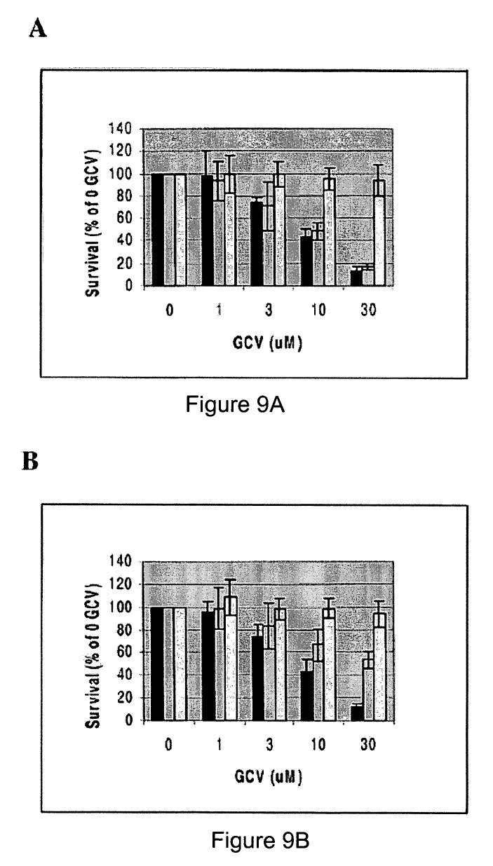

As seen in FIG. 9A and FIG. 9B, the repression of HSV-TK by expression of K2-5F driven by endogenous p53 resulted in a distinct protection against the toxic action of GCV. Thus, in the Saos2/p53-negative cells (FIG. 9A), the presence of the K2-5F-expressing plasmid had no effect on the dose-survival curve for GCV. However, in the Saos 2/p53-positive cells (FIG. 9B), the presence of the K2-5F-expressing plasmid resulted in a substantial protective effect, especially at the higher concentrations of GCV, suggesting that the suicide gene system was regulated by endogenous levels of p53.

### Example 15

### Elimination of Redundant Sequence in the TK promoter (TK-p)

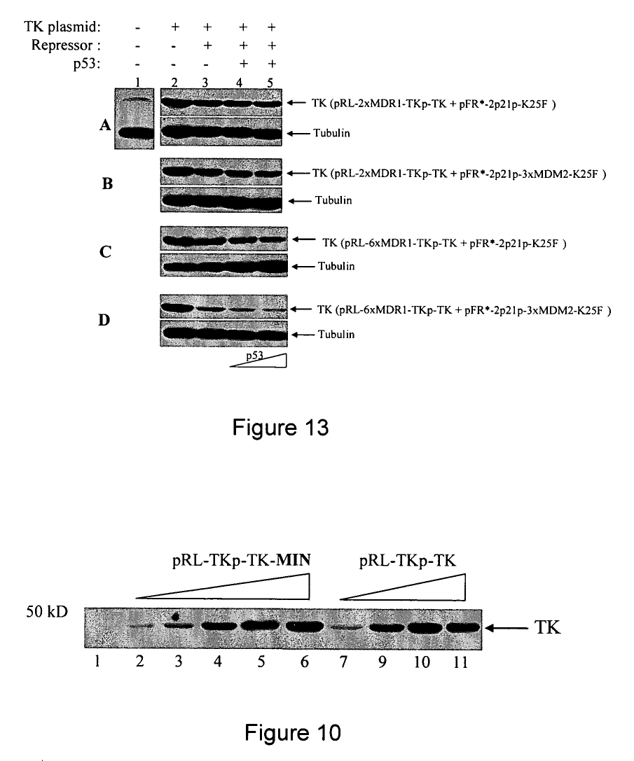

Elimination of redundant or unnecessary sequence from the TK promoter was desired in order to place the nucleic acids and systems of the presently disclosed subject matter into viral vectors for laboratory and therapeutic uses. To accomplish this, the original plasmid pRL-TKp-TK (Promega, Madison, Wis., United States of America) was digested with Bg/II and PvuII followed by end filling the 5′ overhang of Bg/II with T4 DNA polymerase to obtain pRL-Tkp-TK-MIN. This restriction enzyme digestion resulted in reduction of promoter size by 531 upstream bases. The sufficiency and strength of activity of the shortened promoter was checked by Western blot analysis to detect TK expression. As seen in FIG. 10, the expression of TK by the shortened promoter (pRL-Tkp-TK-MIN) was equal in strength to that of the full length promoter (pRL-TKp-TK). The shortened promoter is more suitable for transfer into a viral vector, such as an adenovirus vector or an AAV vector.

### Example 16

### Improved Repression of HSV-TK by Increasing the Number of Repressor Binding Sites

Repression of HSV-TK expression by K2-5F repressor binding was relatively weak in the above Examples. Therefore, improved repression was sought by increasing the number of repressor binding sites available to K2-5F. 2XMDR-1 was PCR amplified from the plasmid pRL-2XMDR-TKp-TK, digested with appropriate restriction enzymes, and sequentially added to the MCS to obtain pRL-6XMDR1-TKp-TK-MIN. The level of HSV-TK expression by each of three plasmids was measured by Western blot.

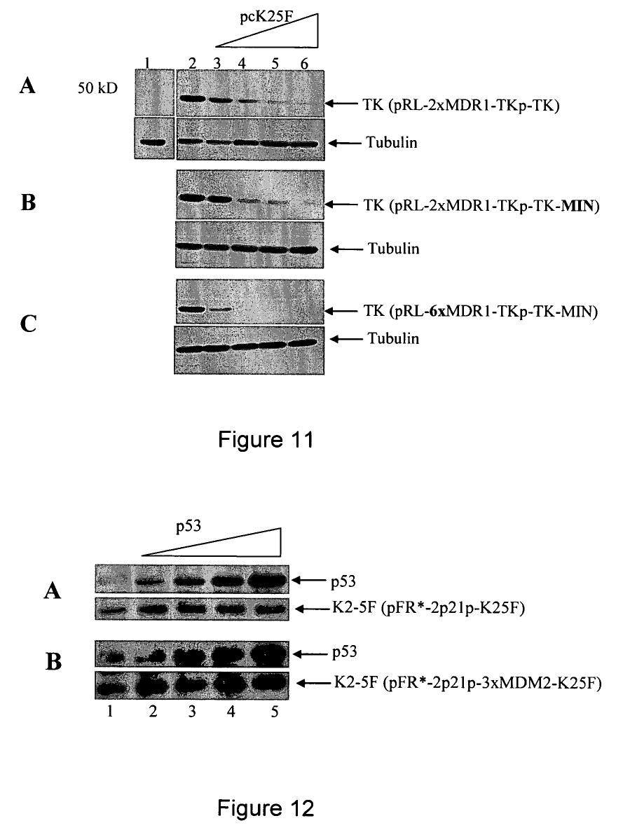

The results are presented in FIG. 11. The plasmids measured were pRL-2XMDR-1-TKp-TK (FIG. 11A), pRL-2XMDR-1-TKp-TK-MIN (FIG. 11B), and pRL-6XMDR-1-TKp-TK-MIN (FIG. 11C). While the two former plasmids showed comparable levels of HSV-TK repression in the presence of the CMV-driven repressor (i.e. pcK2-5F), the third plasmid with three times as many binding sites showed greater repression of HSV-TK expression.

### Example 17

### Increased Expression of K2-5F

Increased expression of K2-5F repressor protein was obtained by increasing the number of p53 binding sites so as to increase p53 driven expression of K2-5F. 2xp21 sites were amplified from pFR*2xp21 p-K2-5F and inserted at HindIII and PstI sites of pFR-luc (Stratagene, La Jolla, Calif., United States of America) to obtain pFR-2p21p-luc. 3xMDM-2 p53 binding sites were obtained by overlap extension PCR and inserted at PstI and XbaI of pFR-2p21 p-luc to obtain pFR-2p21 p-3xMDM2. K2-5F was amplified from pFR*2x21 p-K2-5F and inserted at XbaI of 2p21 p-3XMDM2 to obtain pFR*2p21p-3xMDM2-K2-5F. This increased the number of p53 binding sites on the original repressor expressing plasmid (pFR*-2p21p-K2-5F) by adding three p53 additional binding sequences from the MDM-2 gene to the one binding sequence already on the original repressor expressing plasmid. Each p53 binding sequence from this gene intrinsically contains two individual binding sites.

Thus, the resulting plasmid (pFR*-2p21p-3xMDM2-K24F) has a total of eight p53 binding sites. This new plasmid resulted in increased production of repressor in the presence of p53 as compared to the original plasmid consisting of two p53 binding sites (see FIG. 12). FIG. 12B shows the increased production of K2-5F by pFR*-2p21p-3xMDM2-K2-5F in the presence of increasing amounts of p53 as compared to that produced by pFR*-2p21p-K2-5F, seen in FIG. 12A. This plasmid also caused increased repression of TK when co-transfected with increasing amounts of p53, seen in FIG. 13 as follows. HEK 293T cells were transiently transfected with the following plasmids: control 1.25 μg pcDNA3.1 (lane 1); lanes 2-5 consists of 0.8 μg pRL-2xMDR-1-TKp-TK-MIN (FIGS. 13A and 13B) and 0.8 μg pRL-6xMDR-1-TKp-TK-MIN (FIGS. 13C and 13D). In lanes 3-5, cells were co-transfected with 0.25 μg pFR*-2p21p-K2-5F (FIGS. 13A and 13C), 0.25 μg pFR*-2p21p-3xMDM2-K2-5F (FIGS. 13B and 13D). Lanes 4 and 5 are cotransfected with 0.05 μg and 0.1 μg pCMV-p53. The cumulative effect of increasing repressor binding sites on TK expressing plasmid and increasing p53 binding sites on the repressor plasmid resulted in significantly higher repression of TK.

### Example 18

### Construction of a TGF-β Inducible Promoter

Briefly, a SMAD4-inducible K2-5F repressor and K2-5F-regulated HSV-TK are introduced into cells by transfection. The expression of the transcriptional repressor K2-5F is under the control of a SMAD4-responsive promoter. The modified HSV-TK promoter contains sites that bind the K2-5F repressor.

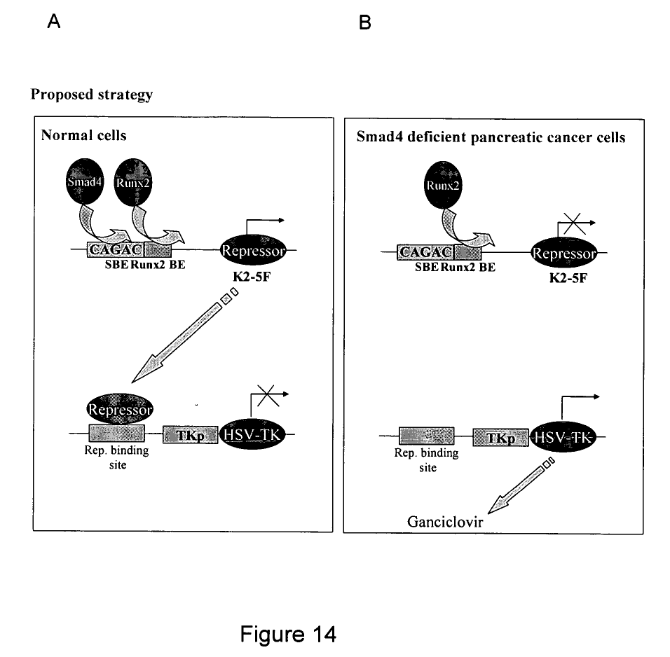

In normal cells (FIG. 14A), wild-type SMAD4 activates the expression of K2-5F, which binds to the TK vector and represses the expression of HSV-TK.

In SMAD4-negative cells (FIG. 14B), there is no K2-5F to inhibit the expression of HSV-TK; thus, these cells make ample enzyme and are susceptible to cell-killing by GCV. Plasmid pCAT-IgCα, which contains the SMAD4/TGF-β inducible promoter was provided by Dr. P. Sideras (Institute for Biomedical Research of the Academy of Athens, Athens, Greece). Plasmid pRK-SMAD4, which is a SMAD4 expression plasmid, was a gift from Dr. R. Derynck (University of Calif. at San Francisco, San Francisco, Calif., United States of America; see Zhang et al., 1996), and mouse Runx2 expression plasmid was obtained from Dr. Stein (University of Massachusetts, Amherst, Mass., United States of America; see Zaidi et al., 2001). The fragment ranging from nucleotide −247 to nucleotide +79 of the promoter was amplified by PCR and inserted at XhoI and HindIII sites of pGL3-basic (Promega Corp., Madison, Wis., United States of America) to obtain pGL3- IgCαB. Luciferase assays were performed by transfecting the respective cells and checking activity with the Luciferase reagent kit (Promega Corp.).

HepG2 liver cells, which are TGF-β sensitive, were acquired from the Tissue Culture Facility at the Lineberger Comprehensive Cancer Center, University of North Carolina at Chapel Hill, Chapel Hill, N.C., United States of America. AsPc-1 (pancreatic SMAD4 negative) cells were acquired from ATCC (American Type Culture Collection, Manassas, Va., United States of America). HepG2 cells were transfected using FuGENE 6 (Roche Diagnostics, Indianapolis, Ind., United States of America). AsPc-1 cells were transfected with LIPOFECTAMINE™ 2000 (Invitrogen, Carlsbad, Calif., United States of America).

The TGF-β inducible plasmid pGL3-IgCαB has an IgCα promoter (B fragment) upstream of the luciferase gene, which provides responsiveness to SMAD4. Co-transfection of the IgCα promoter with an SMAD4 expression plasmid resulted in significant induction of activity, thus showing that the IgCαB promoter is SMAD4 inducible.

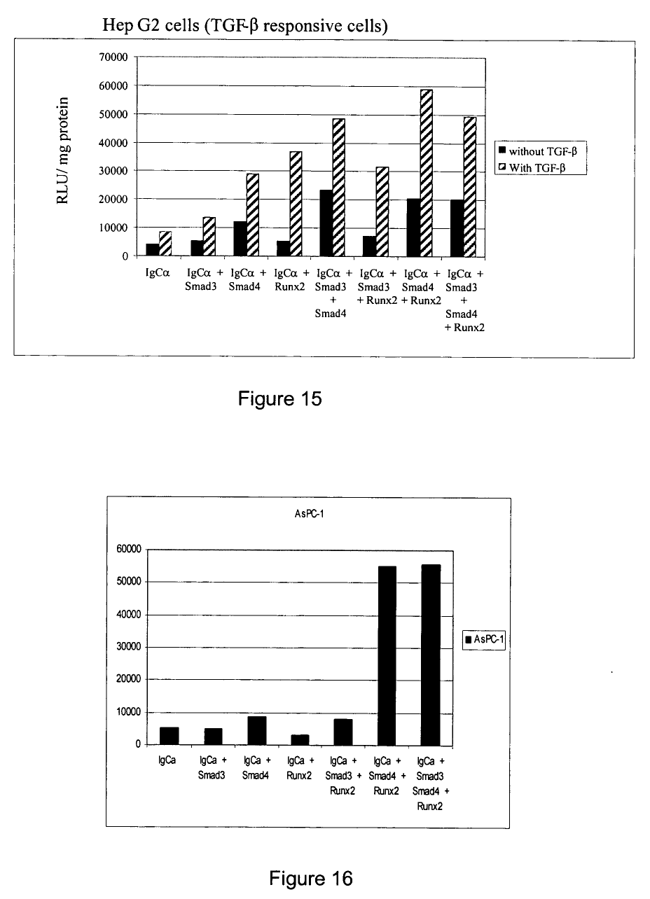

TGF-β responsiveness of IgCα promoter was shown in the following experiment. Hep G2 cells were transiently transfected with 1.0 μg pGL3-IgCα-luciferase under serum starved condition. Cells were co-transfected with 1.0 μg each of plasmids expressing either human SMAD3 or SMAD4 or mouse Runx2 proteins either alone or in various combinations. TGF-β at 400 pMoles was added five hours after transfection. Luciferase activity was determined forty eight hours after transfection. See FIG. 15.

SMAD responsiveness of IgCα promoter was shown in the following experiment. AsPC-1 (SMAD4-negative) cells were transiently transfected with 1.0 μg pGL3-IgCα-luciferase. AsPC-1 cells were co-transfected with 1.0 μg each of plasmids expressing either human SMAD3 or SMAD4 or mouse Runx2 proteins either alone or in various combinations as indicated in FIG. 16. Luciferase activity was determined forty eight hours after transfection.

Under the methods, systems, and kits of the presently disclosed subject matter, the SMAD4-inducible IgCαB promoter is operably linked to the expression of the K2-5F repressor and ultimately controls the expression of HSV-TK, wherein expression of HSV-TK in cells lacking SMAD4 results in the death of the cells.

### REFERENCES

The references listed below as well as all references cited in the specification are incorporated herein by reference to the extent that they supplement, explain, provide a background for or teach methodology, techniques and/or compositions employed herein.

Adelman J P, Hayflick J S, Vasser M & Seeburg P H (1983) In Vitro Deletional Mutagenesis for Bacterial Production of the 20,000-Dalton Form of Human Pituitary Growth Hormone. DNA 2:183-193.

Alam & Cook (1990) Reporter Genes: Application to the Study of Mammalian Gene Transcription. Anal Biochem 188:245-254

Ausubel F M, Brent R, Kingston R E, Moore D D, Seidman J G, Smith J A & Struhl K, eds. (1992) Current Protocols in Molecular Biology. Wiley, New York.

Bartsevich W & Juliano R L (2000) Regulation of the MDR1 Gene by Transcriptional Repressors Selected Using Peptide Combinatorial Libraries. Mol Pharmacol 58(1):1-10.

Beerli R R, Segal D J, Dreier B, Barbas C F 3^{rd} (1998) Toward Controlling Gene Expression at Will: Specific Regulation of the erbB-2/HER-2 Promoter by Using Polydactyl Zinc Finger Proteins Constructed from Modular Building Blocks. Proc Natl Acad Sci USA 95(25):14628-33.

Beerli R R, Dreier B, Barbas C F 3^{rd} (2000a) Positive and Negative Regulation of Endogenous Genes by Designed Transcription Factors. Proc Natl Acad Sci U S A 97(4):1495-500.

Beerli R R, Schopfer U, Dreier B, Barbas C F 3^{rd} (2000b) Chemically Regulated Zinc Finger Transcription Factors. J Biol Chem 275(42):32617-27.

Bischoff J R, Kim D H, Williams A, Heise C, Horn S, Muna M, Ng L, Nye J A, Sampson-Johannes A, Fattaey A & McCormick F (1996) An Adenovirus Mutant That Replicates Selectively in p53-Deficient Human Tumor Cells. Science 274:373-376.

Blancafort P, Segal D J, Barbas C F 3^{rd} (2004) Designing Transcription Factor Architectures for Drug Discovery. Mol Pharmacol 66(6):1361-71.

Brannon-Peppas L & Blanchette J O (2004) Nanoparticle and Targeted Systems for Cancer Therapy. Adv Drug Deliv Rev 56(11): 1649-59.

Cipolla D C, Gonda I, Shak S, Kovesdi I, Crystal R & Sweeney T D (2000) Coarse Spray Delivery to a Localized Region of the Pulmonary Airways for Gene Therapy. Hum Gene Ther 11:361-371.

Cubitt A B, Heim R, Adams S R, Boyd A E, Gross L A & Tsien R Y (1995) Understanding, Improving and Using Green Fluorescent Proteins. Trends Biochem Sci 20:448-455.

El-Aneed A (2004) Current Strategies in Cancer Gene Therapy. Eur J Pharmacol 498(1-3):1-8.

Falke D, Fisher M, Ye D, Juliano R L (2003) Design of Artificial Transcription Factors to Selectively Regulate the Pro-Apoptotic bax Gene. Nucleic Acids Res 31(3): e10.

Felnerova D, Viret J F, Gluck R, Moser C (2004) Liposomes and Virosomes as Delivery Systems for Antigens, Nucleic Acids and Drugs. Curr Opin Biotechnol 15(6):518-29.

Frankel A E, Powell B L, Vallera D A & Neville D M, Jr. (2001) Chimeric Fusion Proteins—Diphtheria Toxin-Based. Curr Opin Investig Drugs 2:1294-1301.

Freeman S M, Whartenby K A, Freeman J L, Abboud C N & Marrogi A J (1996) In Situ Use of Suicide Genes for Cancer Therapy. Semin Oncol 23:31-45.

Glover D M & Hames B D (1995) DNA Cloning : A Practical Approach, 2nd ed. IRL Press at Oxford University Press, Oxford; New York.

Greenberg N M, DeMayo F J, Sheppard P C, Barrios R, Lebovitz R, Finegold M, Angelopoulou R, Dodd J G, Duckworth M L, Rosen J M et al. (1994) The Rat Probasin Gene Promoter Directs Hormonally and Developmentally Regulated Expression of a Heterologous Gene Specifically to the Prostate in Transgenic Mice. Mol Endocrinol 8:230-239.

Habib N A, Hodgson H J, Lemoine N & Pignatelli M (1999) A Phase I/Ii Study of Hepatic Artery Infusion with wtp53-CMV-Ad in Metastatic Malignant Liver Tumours. Hum Gene Ther 10:2019-2034.

Hainaut P (2002) Tumor-Specific Mutations in p53: the Acid Test. Nat Med 8(1):21-3.

Heise C, Sampson-Johannes A, Williams A, McCormick F, Von Hoff D D, Kim D H (1997) ONYX-015, an E1B Gene-Attenuated Adenovirus, Causes Tumor-Specific Cytolysis and Antitumoral Efficacy that can be Augmented by Standard Chemotherapeutic Agents. Nat Med 3(6):639-45.

Heise C C, Williams A, Olesch J, Kim D H (1999a) Efficacy of a Replication-Competent Adenovirus (ONYX-015) Following Intratumoral Injection: Intratumoral Spread and Distribution Effects. Cancer Gene Ther 6(6):499-504.

Heise C C, Williams A M, Xue S, Propst M, Kim D H (1999b) Intravenous Administration of ONYX-015, a Selectively Replicating Adenovirus, Induces Antitu moral Efficacy. Cancer Res 59(11):2623-8.

Hoganson D K, Batra R K, Olsen J C, Boucher R C (1996) Comparison of the Effects of Three Different Toxin Genes and Their Levels of Expression on Cell Growth and Bystander Effect in Lung Adenocarcinoma. Cancer Res 56(6): 1315-23.

Hsieh C S, Macatonia S E, Tripp C S, Wolf S F, O'Garra A & Murphy K M (1993) Development of Th1 Cd4+ T Cells through II-12 Produced by Listeria-Induced Macrophages. Science 260:547-549.

Ichikawa T, Tamiya T, Adachi Y, Ono Y, Matsumoto K, Furuta T, Yoshida Y, Hamada H, Ohmoto T (2000) In Vivo Efficacy and Toxicity of 5-Fluorocytosine/Cytosine Deaminase Gene Therapy for Malignant Gliomas Mediated by Adenovirus. Cancer Gene Ther 7(1):74-82.

Idriss H T & Naismith J H (2000) TNFα and the TNF Receptor Superfamily: Structure-Function Relationship(S). Microsc Res Tech 50:184-195.

Ido A, Uto H, Moriuchi A, Nagata K, Onaga Y, Onaga M, Hori T, Hirono S, Hayashi K, Tamaoki T, Tsubouchi H (2001) Gene Therapy Targeting for Hepatocellular Carcinoma: Selective and Enhanced Suicide Gene Expression Regulated by a Hypoxia-Inducible Enhancer Linked to a Human Alpha-Fetoprotein Promoter. Cancer Res 61 (7):3016-21.

Johannes L & Decaudin D (2005) Protein Toxins: Intracellular Trafficking for Targeted Therapy. Gene Ther [Epub ahead of print].

Kang J S & Kim J S (2000) Zinc Finger Proteins as Designer Transcription Factors. J Biol Chem 275(12):8742-8.

Kijima T, Osaki T, Nishino K, Kumagai T, Funakoshi T, Goto H, Tachibana I, Tanio Y, Kishimoto T (1999) Application of the Cre Recombinase/IoxP System Further Enhances Antitumor Effects in Cell Type-Specific Gene Therapy Against Carcinoembryonic Antigen-Producing Cancer. Cancer Res 59(19):4906-11.

Kim J S, Pabo C O (1997) Transcriptional Repression by Zinc Finger Peptides. Exploring the Potential for Applications in Gene Therapy. J Biol Chem 272(47):29795-800.

Kim J S & Pabo C O (1998) Getting a Handhold on DNA: Design of Poly-Zinc Finger Proteins with Femtomolar Dissociation Constants. Proc Natl Acad Sci U S A 95(6):2812-7.

Kirk C J & Mule J J (2000) Gene-Modified Dendritic Cells for Use in Tumor Vaccines. Hum Gene Ther 11:797-806.

Klohs W D, Fry D W & Kraker A J (1997) Inhibitors of Tyrosine Kinase. Curr Opin Oncol 9:562-568.

Kurihara T, Brough D E, Kovesdi I & Kufe D W (2000) Selectivity of a Replication competent Adenovirus for Human Breast Carcinoma Cells Expressing the MUC1 Antigen. J Clin Invest 106:763-771.

Lane D P & Lain S (2002) Therapeutic Exploitation of the p53 Pathway. Trends Mol Med 8(4 Suppl):S38-42.

Lee S E, Jin R J, Lee S G, Yoon S J, Park M S, Heo D S & Choi H (2000) Development of a New Plasmid Vector with PSA-Promoter and Enhancer Expressing Tissue-Specificity in Prostate Carcinoma Cell Lines. Anticancer Res 20:417-422.

Lin P, Sankar S, Shan S, Dewhirst M W, Polverini P J, Quinn T Q & Peters K G (1998) Inhibition of Tumor Growth by Targeting Tumor Endothelium Using a Soluble Vascular Endothelial Growth Factor Receptor. Cell Growth Differ 9:49-58.

Ling C, Li B, Zhang C, Zhu D, Huang X, Gu W, Li S (2005) Inhibitory Effect of Recombinant Adenovirus Carrying Melittin Gene on Hepatocellular Carcinoma. Ann Oncol. 16(1):109-15.

Loimas S, Toppinen M R, Visakorpi T, Janne J, Wahifors J (2001) Human Prostate Carcinoma Cells as Targets for Herpes Simplex Virus Thymidine Kinase-Mediated Suicide Gene Therapy. Cancer Gene Ther 8(2):137-44.

Lundstrom, K (2004) Gene Therapy Applications of Viral Vectors. Technol Cancer Res Treat 3(5):467-77.

Mackensen A, Lindemann A & Mertelsmann R (1997) Immunostimulatory Cytokines in Somatic Cells and Gene Therapy of Cancer. Cytokine Growth Factor Rev 8:119-128.

Mavria G, Porter C D (2001) Reduced Growth in Response to Ganciclovir Treatment of Subcutaneous Xenografts Expressing HSV-TK in the Vascular Compartment. Gene Ther 8(12):913-20.

Merritt J A, Roth J A, Logothetis C J (2001) Clinical Evaluation of Adenoviral-Mediated p53 Gene Transfer: Review of INGN 201 Studies. Semin Oncol 28(5 Suppl 16):105-14.

Mohr L, Yoon S K, Eastman S J, Chu Q, Scheule R K, Scaglioni P P, Geissler M, Heintges T, Blum H E, Wands J R (2001) Cationic Liposome-Mediated Gene Delivery to the Liver and to Hepatocellular Carcinomas in Mice. Hum Gene Ther 12(7):799-809.

Olsnes, S (2004) The History of Ricin, Abrin and Related toxins. Toxicon 44(4):361-70.

Park B H & Vogelstein B (2003) Tumor-suppressor genes. In: Kufe D W, Pollack R E, Weichselbaum R R, et al., eds. Cancer Medicine. 6th Ed. Lewiston N Y: B C Decker; 2003: 87-106.

Patterson L H (2002) Bioreductively Activated Antitumor N-Oxides: The Case of AQ4N, a Unique Approach to Hypoxia-Activated Cancer Chemotherapy. Drug Metab Rev 34:581-592.

PCT International Publication No. WO 97/47763

Prives C (1998) Signaling to p53: Breaking the MDM2-p53 Circuit. Cell 95(1):5-8.

Rose, M & Botstein D (1983) Construction and Use of Gene Fusions to lacZ (Beta-Galactosidase) that are Expressed in Yeast. Methods Enzymol 101:167-80.

Sambrook J & Russell D W (2001) Molecular Cloning : A Laboratory Manual, 3rd ed. Cold Spring Harbor Laboratory Press, Cold Spring Harbor, N.Y.

Scharfmann R, Axelrod J H & Verma I M (1991) Long-Term in Vivo Expression of Retrovirus-Mediated Gene Transfer in Mouse Fibroblast Implants. Proc Natl Acad Sci U S A 88:46264630.

Shen Y, White E (2001) p53-Dependent Apoptosis Pathways. Adv Cancer Res 82:55-84.

Shibuya M (2001) Structure and Dual Function of Vascular Endothelial Growth Factor Receptor-1 (Flt-1). Int J Biochem Cell Biol 33:409-420

Silhavy T J, Berman M L, Enquist L W & Cold Spring Harbor Laboratory. (1984) Experiments with Gene Fusions. Cold Spring Harbor Laboratory, Cold Spring Harbor, N.Y.

Siders W M, Halloran P J, Fenton R G (1998) Melanoma-Specific Cytotoxicity Induced by a Tyrosinase Promoter-Enhancer/Herpes Simplex Virus Thymidine Kinase Adenovirus. Cancer Gene Ther 5(5):281 -91.

Srivastava R K (2001) Trail/Apo-21: Mechanisms and Clinical Applications in Cancer. Neoplasia 3:535-546.

Tamura M, Ikenaka K, Tamura K, Yoshimatsu T, Miyao Y, Kishima H, Mabuchi E, Shimizu K (1998) Transduction of Glioma Cells Using a High-Titer Retroviral Vector System and Their Subsequent Migration in Brain Tumors. Gene Ther5(12):1698-704.

Thomas M, Pim D, Banks L (1999) The role of the E6-p53 interaction in the molecular pathogenesis of HPV. Oncogene 18(53):7690-700.

U.S. Pat. No. 5,858,784

U.S. Pat. No. 6,013,638

U.S. Pat. No. 6,022,737

U.S. Pat. No. 6,136,295

U.S. Pat. No. 6,458,587

U.S. Pat. No. 6,489,162

U.S. Pat. No. 6,491,907

U.S. Pat. No. 6,548,286

Vassaux G & Lemoine N R (2000) Gene Therapy for Carcinoma of the Breast: Genetic Toxins. Breast Cancer Res 2(1):22-7.

Vijayanathan V, Thomas T, Thomas T J (2002) DNA Nanoparticles and Development of DNA Delivery Vehicles for Gene Therapy. Biochemistry 41 (48):14085-94.

Walther W & Stein U (1999) Therapeutic Genes for Cancer Gene Therapy. Mol Biotechnol 13:21-28.

Williams R S, Thomas J A, Fina M, German Z & Benjamin I J (1993) Human Heat Shock Protein 70 (Hsp70) Protects Murine Cells from Injury During Metabolic Stress. J Clin Invest 92:503-508.

Wu X & Deng Y (2002) Bax and BH3-Domain-Only Proteins in p53-Mediated Apoptosis. FrontBiosci 7:d151-6.

Xu D, Ye D, Fisher M, Juliano R L (2002) Selective Inhibition of P-Glycoprotein Expression in Multidrug-Resistant Tumor Cells by a Designed Transcriptional Regulator. J Pharmacol Exp Ther 302(3):963-71.

Yazawa K, Fisher W E & Brunicardi F C (2002) Current Progress in Suicide Gene Therapy for Cancer. World J Surg 26:783-789.

Yu D C, Chen Y, Seng M, Dilley J & Henderson D R (1999) The Addition of Adenovirus Type 5 Region E3 Enables Calydon Virus 787 to Eliminate Distant Prostate Tumor Xenografts. Cancer Res 59:4200-4203.

Zaidi S K, Javed A, Choi J Y, van Wijnen A J, Stein J L, Lian J B, Stein G S (2001) A Specific Targeting Signal Directs Runx2/Cbfa1 to Subnuclear Domains and Contributes to Transactivation of the Osteocalcin Gene. J Cell Biol 114: 3093-3102.

Zhang Y, Feng X, We R, Derynck R (1996) Receptor-Associated Mad Homologues Synergize as Effectors of the TGF-beta Response. Nature 383(6596):168-72.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A method for selectively conferring toxicity on a cancerous cell, the method comprising providing to a cell:
(a) a first nucleic acid encoding a first gene product that is capable of conferring toxicity to a cell in which it is expressed; and
(b) a second nucleic acid encoding a second gene product that protects non-cancerous cells from developing toxicity by substantially repressing expression of the first gene product and wherein expression of the second gene product is induced by a protein functionally present in non-cancerous cells but substantially functionally absent in cancerous cells, thereby allowing expression of the first gene product substantially only in cancerous cells.

2. The method of claim 1, wherein the toxicity comprises a chemosensitivity, the method further comprising contacting the cell with a chemical agent that interacts with the first gene product, and further wherein an interaction between the chemical agent and the first gene product produces a converted chemical compound that is toxic to the cell, thereby selectively killing cancerous cells having chemosensitivity to the chemical agent.

3. The method of claim 1, the method further comprising providing to the cell a drug-responsive system that enhances the conference of toxicity to a cancerous cell or enhances protection of a non-cancerous cell from toxicity.

4. The method of claim 1, wherein the gene encoded by the first nucleic acid is selected from the group consisting of TNF-α, TNF-β, FasL, APRIL, BAFF, OPGL/TRANCE/RANCL, TALL-1, TRAIL, Bad, Bax, Bak, Bcl-x_{S}, Bok/Mtd, Bcl-G_{L}, Bik/Nbk, Blk, Bid, Hrk/DP5, Bim/Bod, Bmf, Noxa, Puma/Bbc-3, C. elegans Egl-1, ricin, abrin, Diptheria toxin, and mellitin.

5. The method of claim 2, wherein:
i. the first gene product is an HSV-TK polypeptide and the chemical agent is ganciclovir, or
ii. the first gene product is a cytosine deaminase polypeptide and the chemical agent is 5-fluorocytosine; or
iii. the first gene product is a nitroreductase polypeptide and the chemical agent is CB1954; or
iv. the first gene product is a carboxypeptidase polypeptide and the chemical agent is CMDA.

6. The method of claim 1, wherein the second gene product comprises at least one repressor domain and at least two DNA binding domains.

7. The method of claim 6, wherein the at least two DNA binding domains comprise zinc finger binding domains.

8. The method of claim 1, wherein the second gene product is a K2-5F polypeptide.

9. The method of claim 1, wherein the protein functionally present in non-cancerous cells but substantially functionally absent in cancerous cells is selected from the group consisting of a p53 protein, an RB1 protein, an SMAD4 protein, a WT1 protein, and combinations thereof.

10. The method of claim 1, wherein the first nucleic acid is provided in a first vector and the second nucleic acid is provided in a second vector.

11. The method of claim 1, wherein the first nucleic acid and the second nucleic acid are provided in a single vector.

12. The method of claim 1, wherein the first nucleic acid and the second nucleic acid are provided to the cell in a viral or a nonviral gene delivery vehicle.

13. The method of claim 1, wherein the cell is a mammalian cell.

14. The method of claim 1, wherein the cell is present within a mammal selected from the group consisting of rodents, non-human primates, and humans.

15. A system for selectively conferring toxicity to cancerous cells, the system comprising:
(a) a first nucleic acid encoding a first gene product that is capable of conferring toxicity to a cell in which it is expressed; and
(b) a second nucleic acid encoding a second gene product that protects non-cancerous cells from developing toxicity by substantially repressing expression of the first gene product and wherein expression of the second gene product is induced by a protein functionally present in non-cancerous cells but substantially functionally absent in cancerous cells.

16. The system of claim 15, wherein the toxicity comprises a chemosensitivity, the system further comprising a chemical agent that interacts with the first gene product, and further wherein an interaction between the chemical agent and the first gene product produces a converted chemical compound that is toxic to the cell, thereby selectively killing cancerous cells having chemosensitivity to the chemical agent.

17. The system of claim 15, further comprising a pharmaceutically or physiologically acceptable carrier.

18. A kit for selectively conferring toxicity to cancerous cells, the kit comprising the system of claim 15.

19. The system of claim 16, the method further comprising a drug-responsive system that enhances the conference of toxicity to a cancerous cell or enhances protection of a non-cancerous cell from toxicity.

20. The system of claim 15, wherein the gene encoded by the first nucleic acid is selected from the group consisting of TNF-α, TNF-β, FasL, APRIL, BAFF, OPGL/TRANCE/RANCL, TALL-1, TRAIL, Bad, Bax, Bak, Bcl-x_{S}, Bok/Mtd, Bcl-G_{L}, Bik/Nbk, Blk, Bid, Hrk/DP5, Bim/Bod, Bmf, Noxa, Puma/Bbc-3, C. elegans Egl-1, ricin, abrin, Diptheria toxin, and mellitin.

21. The system of claim 16, wherein:
i. the first gene product is an HSV-TK polypeptide and the chemical agent is ganciclovir, or
ii. the first gene product is a cytosine deaminase polypeptide and the chemical agent is 5-fluorocytosine; or
iii. the first gene product is a nitroreductase polypeptide and the chemical agent is CB1954; or
iv. the first gene product is a carboxypeptidase polypeptide and the chemical agent is CMDA.

22. The system of claim 16, wherein the second gene product comprises at least one repressor domain and at least two DNA binding domains.

23. The system of claim 22, wherein the at least two DNA binding domains comprise zinc finger binding domains.

24. The system of claim 15, wherein the second gene product is a K2-5F polypeptide.

25. The system of claim 15, wherein the protein functionally present in non-cancerous cells but substantially functionally absent in cancerous cells is selected from the group consisting of a p53 protein, an RB1 protein, an SMAD4 protein, a WT1 protein, and combinations thereof.

26. The system of claim 15, wherein the first nucleic acid is provided in a first vector and the second nucleic acid is provided in a second vector.

27. The system of claim 15, wherein the first nucleic acid and the second nucleic acid are provided in a single vector.

28. The system of claim 15, wherein the first nucleic acid and the second nucleic acid are provided in a viral or a nonviral gene delivery vehicle.
